(19) United States
(12) Patent Application Publication
(10) Pub. No.: US 2026/0007716 A1
(43) Pub. Date: Jan. 8, 2026

(54) DESLORELIN FOR ATTENUATION OF ANIMAL URINE ODOR

(71) Applicant: VIRBAC, Carros (FR)

(72) Inventors: Dorine DESPOSITO, Colomars (FR); Jean-Baptiste RASCLE, Villeneuve-Loubet (FR); Françoise EMILY, Roquebillière (FR); Christelle FONTAINE, La Roquette-sur-Var (FR)

(73) Assignee: VIRBAC, Carros (FR)

(21) Appl. No.: 18/879,928

(22) PCT Filed: Jun. 29, 2023
(86) PCT No.: PCT/EP2023/067885

§ 371 Date: Dec. 30, 2024

Related U.S. Application Data
(60) Provisional application No. 63/357,223, filed on Jun. 30, 2022.

(51) Int. Cl. A61K 38/09; A61P 13/02

(52) U.S. Cl. CPC A61K 38/09; A61P 13/02

(57) ABSTRACT

Deslorelin for its use in attenuation or reduction of urine and/or feces odor of a non-human animal, preferably a mammal, preferentially in a companion animal. In particular, a process for attenuating or reducing urine and/or feces odor in a non-human animal including a step of administering to the non-human animal a quantity of deslorelin such that the deslorelin plasma concentration in the non-human animal is higher than 10 pg/mL. Also, a process for down regulating levels of molecules in urine and/or feces of a non-human animal, which includes administering to the non-human animal a quantity of deslorelin.

### TECHNICAL FIELD

The present disclosure generally relates to pharmaceutical compositions of deslorelin for use in attenuation of urine and feces odor of a non-human animal, preferably a mammal, preferentially in a companion animal.

### BACKGROUND

Deslorelin is a gonadotropin analog of gonadotropin-releasing hormone (GnRH) and acts by suppressing gonadopituitary axis function when given in low continuous doses.

GnRH is a hypothalamic decapeptide, which acts at the top of the cascade that coordinates the function of the hypothalamic-pituitary gonadal axis. GnRH will bind to GnRH receptors (7 transmembrane receptors) causing the pituitary to produce and release two key gonadotropins, follicle stimulating hormone (FSH) and luteinizing hormone (LH), which in turn control gonadal function
LH (Luteinizing Hormone), in addition to its role in gametogenesis, promotes the production of sex steroids (androgens, estrogens and progesterone);
FSH (Follicle Stimulating Hormone) stimulates the production of gametes (spermatozoa and eggs).

When GnRH binds to the receptors, it increases the synthesis of the mRNA of its own receptor and raises the sensitivity of the pituitary to GnRH. A continuous secretion of GnRH has an opposite effect (a collapse of LH and FSH secretion is observed: one can speak of desensitization). (Finch A. R. et al., Agonist-induced internalization and downregulation of gonadotropin-releasing hormone receptors. Am. J. Physiol. Cell Physiol. 2009 September, 297 (3)).

Pituitary desensitization to GnRH is triggered by internalization of GnRH receptors (which are no longer present to bind to the cell surface) and inactivation of the intracellular signaling cascade. Once desensitization of pituitary cells to GnRH is initiated, LH concentrations fall to undetectable levels and thus fail to support testosterone and sperm production.

In order to solve a contraceptive problem in animals, some people had the idea to use GnRH agonists continuously and over a long period of time as a contraceptive. This is how the idea of designing a deslorelin implant that continuously diffuses a GnRH agonist (deslorelin) came about.

This type of implant is used in 2 different ways:
Fertility induction (short term implant): Ovuplant
Induction of infertility by long-term action: Suprelorin

It is known in the state of the art that these agonist-based implants have two phases of action, during the injection of the implant, there is a release of deslorelin which will induce a strong stimulation of the LH and FSH production cascade, this step is called “flare-up”. In male dogs, this induces a production of LH and FSH leading to a strong synthesis of testosterone. The males are then super fertile (improved sperm quality, improved motility).

After this “flare-up” period, a phase of infertility is established with adverse effects on all aspects of sperm quality and testosterone concentration (Goericke-Pesch S., Long-term effects of GnRH agonists on fertility and behaviour., Reprod Domest Anim., 2017, 52 Suppl 2:336-347)

In addition to being tested in dogs, studies have been conducted in cats and pussies (Fontaine C., Long-term contraception in a small implant: A review of Suprelorin (deslorelin) studies in cats., J Feline Med Surg., 2015, 17 (9), pages 766-771). This shows the effectiveness of the implant as a contraceptive in both males and females. During the period of fertility suppression, steroid hormone concentrations below 1 ng/ml for progesterone and 10 pg/ml for estradiol were measured in females; in male cats, plasma testosterone concentration remained at basal levels (<0.1 ng/ml). The duration of action with a 4.7 mg implant in males was 78.8±12.9 weeks and ranged from 61.7 weeks (approximately 15 months) to 100.7 weeks (approximately 25 months).

Until now, the known data did not show a PK/PD (pharmacokinetic/pharmacodynamic) relationship and the efficacy of the product was explained by the very large burst shortly after implantation and a very long duration of GNRH receptors saturation. The current RCP data in dogs indicate a presence of deslorelin for 2.5 months while the effect is observed for at least 6 months (source: EMA website: https://www.ema.europa.eu/en/documents/product-information/suprelorin-epar-product-information_en.pdf).

New data have been generated and are showing that deslorelin can be measured as long as the effect is observed in dogs and cats males and females.

In addition to this, it is shown that maintaining a range of concentration of deslorelin in the blood stream to obtain and keep the effects of castration is linked to PK/PD interaction. Contrary to the prior art and the common belief concerning the mode of action of Deslorelin, it is moreover clearly demonstrated a PK/PD relationship between the in vivo levels of Deslorelin and the PD effects, allowing us to identify an efficacy threshold.

Besides the contraceptive effect of deslorelin, other effects have been noticed as reported in the examples illustrating the present invention on sexual behaviors and urine marking and/or urine odor.

Urine marking is the normal and deliberate deposition of urine as a:
Pheromonal signal to other animals; especially unneutered male cats
Territorial signal
Sign of stress or arousal related to the social or physical environment.

In this context, urine may also have a pungent odor and it is desirable, especially for the pet owner, to eliminate or mask and/or mitigate or reduce this odor. Several commercially available enzymatic urine odor neutralizers that should be used on surfaces after washing the area with mild detergent can be used for removing the odor but they are fastidious. Neutering or spaying is a proven treatment particularly for cats who mark as a reproductive or territorial advertisement. One of the reason for surgical castration is to alleviate the strong and persistent urine odor in male animal, particularly with cat and ferret. It is known that after surgical castration, the urine odor is improved in few days.

However, using surgery to solve this problem is much more traumatic considering the cause of said problem and can lead to adverse effects.

One of the purpose of the present application is to show that deslorelin (for example when administered in an implant such as Suprelorin®) administration decreases urine and/or feces odor. Surprisingly, it has been shown by the inventors that odor reduction is mediated via at list two pathways. Indeed, one such pathway is the testosterone-mediated sex hormone pathway. The inventors have also demonstrated at list another pathway unrelated to sex hormones as described in the present application, and shown an improved decrease in the unpleasant odor of urine and/or feces.

Hence, the problem is solved by using deslorelin which attenuates urine and/or feces odor in inducing a chemical castration of administrated non-human animal.

A recent study conducted by Chumki Banik et al. (Simultaneous Chemical and Sensory Analysis of Domestic Cat Urine and Feces with Headspace Solid-Phase Microextraction and GC-MS-Olfactometry Separations 2021, 8, 15 pp 1 of 19) had the objective to identify the volatile organic chemicals (VOCs) and associated odors in cat urine and feces using gas chromatography-mass spectrometry and simultaneous sensory analysis of fresh and aged samples. The objective of this study was different from the present invention.

### SUMMARY OF INVENTION

The present invention is relating to deslorelin for its use in attenuating or reducing urine and/or feces odor in a non-human animal, in particular unpleasant odor, wherein after administration to said non-human animal, the deslorelin plasma concentration of said non-human animal is higher than 10 pg/mL. In a preferred embodiment, the deslorelin plasma concentration is between 10 pg/mL and 400 pg/ml, from 8 days after administration up to at least 6 months. In another preferred embodiment, deslorelin is administered or released after administration continuously in said non-human animal. For instance, the deslorelin is released substantially continuously from 8 days after administration to at least 6 months at a level plasma concentration below 400 pg/mL.

In another preferred embodiment, after administration, deslorelin modulates, i.e. down regulates or up regulates the molecules responsible of urine and/or feces odor, said molecules being selected from the group comprising felinine derivative compounds, the volatile organic compounds (VOCs), the sulfur compounds and the testosterone induced compounds.

In a particular embodiment, deslorelin modulates, i.e. down regulates or up regulates molecules responsible of urine and/or feces odor selected from:
sulfur compounds selected from the group comprising: 4-methyl-4-sulfanyl-pentan-2-one; 3-methyl-3-sulfanyl-butan-1-ol; 3-methyl-3-sulfanyl-1-butyl formate;
VOCs: 2,5-dimethyl pyrazine; 1,2-dichloro-propane; 4,4-dimethyl-2-pentanone; and 3-methyl-2-buten-1-ol;
   compounds selected from the group comprising: 2-(methylthio)-1-ethanol; 2-methyl-3-furanthiol; oxathiolane; 4-(methylthio)-2-butanone; 3-methylbutyl-3-sulfanyl formate; and 3-methyl-3-(methylthio)-1-butanol;
   paracresol;
   felinine and derivatives thereof such as: 3-mercapto-3-methyl-1-butanol; 3-mercapto-3-methylbutyl formate; 3-methyl-3-methylthio-1-butanol; and 3-methyl-3-(2-methyldisulfanyl)-1-butanol;
   felinine precursors such as: N-acetylfelinine, Y-glutamylfelinylglycine and 3-methylbutanolcysteinylglycine;
cauxine;
2-phenylethylamine, and S-(1-hydroxy-3,7-dimethyl-6-octen-3-yl)cysteine;
isovalthine;
isobuteine;
trimethylamine;
indole;
cadaverine and cadaverine derivatives such as N-acetylcadaverine (also known as acetylcadaverine), and glutathionylaminopropylcadaverine,
short-chain free fatty acids such as: acetic acid, propanoic acid, 2-methylpropanoic acid, butanoic acid, 3-methylbutanoic acid, and pentanoic acid;
crotonic acid;
putrescine and derivatives thereof such as N-acetylputrescine, and p-coumaroylputrescine;
   mercapto derivatives such as 2-mercapto-3-butanone, 2-mercaptoethanol, 3-mercaptohexyl butyrate, mercaptopyruvic acid, 3-mercaptopropionic acid, 3-mercapto-3-methylbutan-1-ol, 7-mercaptoheptanoylthreonine, 1alpha,5alpha-dimercaptoandrostane-3alpha,17beta-diol, mercaptopyruvic acid and 2-mercapto-2-methylpentan-4-one; and
taurine
valine.

In a more preferred embodiment, the molecules responsible of urine and/or feces odor on which deslorelin has an effect are selected from:
sulfur compounds selected from the group comprising: 4-methyl-4-sulfanyl-pentan-2-one; 3-methyl-3-sulfanyl-butan-1-ol; 3-methyl-3-sulfanyl-1-butyl formate;
VOCs: 2,5-dimethyl pyrazine; 1,2-dichloro-propane; 4,4-dimethyl-2-pentanone; and 3-methyl-2-buten-1-ol;
compounds selected from the group comprising: 2-(methylthio)-1-ethanol; 2-methyl-3-furanthiol; oxathiolane; 4-(methylthio)-2-butanone; 3-methylbutyl-3-sulfanyl formate; and 3-methyl-3-(methylthio)-1-butanol;
paracresol
felinine and derivatives: 3-mercapto-3-methyl-1-butanol; 3-mercapto-3-methylbutyl formate; 3-methyl-3-methylthio-1-butanol; and 3-methyl-3-(2-methyldisulfanyl)-1-butanol;
felinine precursors: N-acetylfelinine, Y-glutamylfelinylglycine et 3-methylbutanolcysteinylglycine;
cauxine;
   2-phenylethylamine, and S-(1-hydroxy-3,7-dimethyl-6-octen-3-yl)cysteine;
isovalthine;
isobuteine;
trimethylamine,
indole,
cadaverine and cadaverine derivatives such as N-acetylcadaverine, and glutathionylaminopropylcadaverine;
short-chain free fatty acids such as: acetic acid, propanoic acid, 2-methylpropanoic acid, butanoic acid, 3-methylbutanoic acid, and pentanoic acid;
crotonic acid; and
putrescine and derivatives thereof such as N-acetylputrescine, and p-coumaroylputrescine;
   the mercapto derivatives like 2-mercapto-3-butanone, 2-Mercaptoethanol, 3-Mercaptohexyl butyrate, Mercaptopyruvic acid, 3-Mercaptopropionic acid, 3-Mercapto-3-methylbutan-1-ol, 7-Mercaptoheptanoyl threonine, 1 alpha,5alpha-Dimercaptoandrostane-3alpha,17beta-diol, Mercaptopyruvic acid;
Taurine.

In an even more preferred embodiment, the molecules are selected from the list of:
trimethylamine,
3-methyl butanoic acid
Indole
   cadaverine and derivatives like acetylcadaverine, Glutathionylaminopropylcadaverine,
Short-chain free fatty acids: acetic acid, propanoic acid, 2-methylpropanoic acid, butanoic acid, 3-methylbutanoic acid, and pentanoic acid
Crotonic acid
   Putrescine and derivatives like N-acetylputrescine, p-coumaroylputrescine;
   the mercapto derivatives like 2-mercapto-3-butanone, 2-Mercaptoethanol, 3-Mercaptohexyl butyrate, Mercaptopyruvic acid, 3-Mercaptopropionic acid, 3-Mercapto-3-methylbutan-1-ol, 7-Mercaptoheptanoylthreonine, 1alpha,5alpha-Dimercaptoandrostane-3alpha,17beta-diol, Mercaptopyruvic acid;
Taurine.

According to another preferred embodiment, deslorelin down regulates molecules responsible of urine and/or feces odor, said molecules being selected from the group comprising:
sulfur compounds selected from the group comprising: 4-methyl-4-sulfanyl-pentan-2-one; 3-methyl-3-sulfanyl-butan-1-ol; 3-methyl-3-sulfanyl-1-butyl formate;
VOCs: 2,5-dimethyl pyrazine; 1,2-dichloro-propane; 4,4-dimethyl-2-pentanone; and 3-methyl-2-buten-1-ol;
compounds selected from the group comprising 2-(methylthio)-1-ethanol; 2-methyl-3-furanthiol; oxathiolane; 4-(methylthio)-2-butanone; 3-methylbutyl-3-sulfanyl formate; and 3-methyl-3-(methylthio)-1-butanol;
paracresol
felinine and derivatives thereof such as: 3-mercapto-3-methyl-1-butanol; 3-mercapto-3-methylbutyl formate; 3-methyl-3-methylthio-1-butanol; and 3-methyl-3-(2-methyldisulfanyl)-1-butanol;
felinine precursors such as: N-acetylfelinine, γ-glutamylfelinylglycine and 3-methylbutanolcysteinylglycine;
cauxine;
   2-phenylethylamine, and S-(1-hydroxy-3,7-dimethyl-6-octen-3-yl)cysteine;
isovalthine;
isobuteine;
trimethylamine;
indole;
   cadaverine and derivatives like N-acetylcadaverine, and glutathionylaminopropylcadaverine;
short-chain free fatty acids such as: acetic acid, propanoic acid, 2-methylpropanoic acid, butanoic acid, 3-methylbutanoic acid, and pentanoic acid;
   crotonic acid;
putrescine and derivatives thereof such as N-acetylputrescine, and p-coumaroylputrescine;
   mercapto derivatives such as 2-mercapto-3-butanone, 2-mercaptoethanol, 3-mercaptohexyl butyrate, mercaptopyruvic acid, 3-mercaptopropionic acid, 3-mercapto-3-methylbutan-1-ol, 7-mercaptoheptanoylthreonine, 1 alpha,5alpha-Dimercaptoandrostane-3alpha,17beta-diol, mercaptopyruvic acid; 2-mercapto-2-methylpentan-4-one; and
taurine.

According to another preferred embodiment, deslorelin up regulates molecules responsible of urine and/or feces odor, said molecules being selected from the group comprising 2-mercapto 2-methylpentan-4-one and valine.

According to another embodiment, the invention relates to the non-pharmaceutical or non-therapeutical use of deslorelin to modulate, i.e. to down regulate or up regulate the levels of molecules in urine and or feces of a non-human animal, said molecules being selected from the group comprising: of
sulfur compounds selected from the group comprising: 4-methyl-4-sulfanyl-pentan-2-one; 3-methyl-3-sulfanyl-butan-1-ol; 3-methyl-3-sulfanyl-1-butyl formate;
VOCs: 2,5-dimethyl pyrazine; 1,2-dichloro-propane; 4,4-dimethyl-2-pentanone; and 3-methyl-2-buten-1-ol;
compounds selected from the group comprising: 2-(methylthio)-1-ethanol; 2-methyl-3-furanthiol; oxathiolane; 4-(methylthio)-2-butanone; 3-methylbutyl-3-sulfanyl formate; and 3-methyl-3-(methylthio)-1-butanol;
paracresol;
felinine and derivatives: 3-mercapto-3-methyl-1-butanol; 3-mercapto-3-methylbutyl formate; 3-methyl-3-methylthio-1-butanol; 3-methyl-3-(2-methyldisulfanyl)-1-butanol, and 3-mercapto-3-methyl-1-butanol.
   felinine precursors such as: N-acetylfelinine, γ-glutamylfelinylglycine and 3-methylbutanolcysteinylglycine;
cauxine;
2-phenylethylamine, and S-(1-hydroxy-3,7-dimethyl-6-octen-3-yl)cysteine;
isovalthine;
isobuteine;
trimethylamine;
indole;
   cadaverine and cadaverine derivatives such as: acetylcadaverine, and glutathionylaminopropylcadaverine;
   short-chain free fatty acids such as: acetic acid, propanoic acid, 2-methylpropanoic acid, butanoic acid, 3-methylbutanoic acid, and pentanoic acid;
   crotonic acid
   putrescine and derivatives thereof such as N-acetylputrescine, and p-coumaroylputrescine;
      mercapto derivatives such as: 2-mercapto-3-butanone, 2-mercaptoethanol, 3-mercaptohexyl butyrate, mercaptopyruvic acid, 3-mercaptopropionic acid, 3-mercapto-3-methylbutan-1-ol, 7-mercaptoheptanoylthreonine, 1alpha,5alpha-dimercaptoandrostane-3alpha,17beta-diol, mercaptopyruvic acid and 2-mercapto-2-methylpentan-4-one,
taurine, and
valine.

In a preferred embodiment, the non-pharmaceutical use or non-therapeutical use of deslorelin is to modulate, i.e. to down regulate or up regulate the levels of molecules in a non-human animal, said molecules being selected from the group comprising:
trimethylamine,
3-methyl butanoic acid,
indole,
   cadaverine and cadaverine derivatives such as acetylcadaverine, and glutathionylaminopropylcadaverine,
   short-chain free fatty acids such as: acetic acid, propanoic acid, 2-methylpropanoic acid, butanoic acid, 3-methylbutanoic acid, and pentanoic acid,
crotonic acid,
putrescine and derivatives thereof such as N-acetylputrescine, and p-coumaroylputrescine;
   mercapto derivatives such as 2-mercapto-3-butanone, 2-mercaptoethanol, 3-mercaptohexyl butyrate, mercaptopyruvic acid, 3-mercaptopropionic acid, 3-mercapto-3-methylbutan-1-ol, 7-mercaptoheptanoylthreonine, and 1alpha,5alpha-dimercaptoandrostane-3alpha,17beta-diol, mercaptopyruvic acid and 2-mercapto-2-methylpentan-4-one; and
taurine.

In a still preferred embodiment, the deslorelin modulates, i.e. down regulates or up regulates the levels of molecules in urine.

In a preferred embodiment yet, the deslorelin modulates, i.e. down regulates or up regulates the levels of molecules in feces.

In a still preferred embodiment, the invention relates to the non therapeutical use of deslorelin to down regulate the levels of molecules in urine and or feces of a non-human animal, said molecules being selected from the group comprising:
sulfur compounds selected from the group comprising: 4-methyl-4-sulfanyl-pentan-2-one; 3-methyl-3-sulfanyl-butan-1-ol; 3-methyl-3-sulfanyl-1-butyl formate;
VOCs: 2,5-dimethyl pyrazine; 1,2-dichloro-propane; 4,4-dimethyl-2-pentanone; and 3-methyl-2-buten-1-ol;
compounds selected from the comprising 2-(methylthio)-1-ethanol; 2-methyl-3-furanthiol; oxathiolane; 4-(methylthio)-2-butanone; 3-methylbutyl-3-sulfanyl formate; and 3-methyl-3-(methylthio)-1-butanol;
paracresol;
felinine and derivatives thereof such as: 3-mercapto-3-methyl-1-butanol; 3-mercapto-3-methylbutyl formate; 3-methyl-3-methylthio-1-butanol; and 3-methyl-3-(2-methyldisulfanyl)-1-butanol;
felinine precursors such as: N-acetylfelinine, γ-glutamylfelinylglycine and 3-methylbutanolcysteinylglycine;
cauxine;
2-phenylethylamine, and S-(1-hydroxy-3,7-dimethyl-6-octen-3-yl)cysteine;
isovalthine;
isobuteine;
trimethylamine;
indole;
cadaverine and cadaverine derivatives such as N-acetylcadaverine, and glutathionylaminopropylcadaverine;
short-chain free fatty acids such as: acetic acid, propanoic acid, 2-methylpropanoic acid, butanoic acid, 3-methylbutanoic acid, and pentanoic acid;
crotonic acid;
putrescine and derivatives thereof such as N-acetylputrescine, and p-coumaroylputrescine;
   mercapto derivatives such as 2-mercapto-3-butanone, 2-mercaptoethanol, 3-mercaptohexyl butyrate, mercaptopyruvic acid, 3-mercaptopropionic acid, 3-mercapto-3-methylbutan-1-ol, 7-mercaptoheptanoylthreonine, 1alpha,5alpha-dimercaptoandrostane-3alpha,17beta-diol, and mercaptopyruvic acid; and
taurine.

In another preferred embodiment, the invention relates to the non-therapeutical use of deslorelin to up regulate the levels of molecules in urine and/or feces of a non-human animal, said molecules being selected from the group comprising 2-mercapto 2-methylpentan-4-one and valine.

The invention also relates to a process for attenuating or reducing urine and/or feces odor in a non-human animal, the process consisting in administering to the non-human animal a quantity of deslorelin wherein after administration to said non-human animal, the deslorelin plasma concentration of said non-human animal is higher than 10 pg/mL.

Another object of the invention is also a process for attenuating or reducing urine and/or feces odor in a non-human animal, the process consisting in administering to the non-human animal a quantity of deslorelin that will induce modulations, i.e. down regulations or up regulations, of the levels of odorifying molecules in urine and/or feces. Preferably, the non-human animal is an intact male cat. According to this embodiment, deslorelin induces modulations, i.e. down regulations or up regulations, of the levels of odorifying molecules in urine and/or feces via sex hormone synthesis pathway.

In another embodiment, the invention also relates to a process for improving the attenuation or reduction of the urine and/or feces odor in a non-human intact male animal compared to a neutered male, the process consisting in administering to the non-human intact male animal a quantity of deslorelin that induces modulations, i.e. down regulations or up regulations, of the levels of odorifying molecules in urine and/or feces. According to this embodiment, deslorelin induces modulations, i.e. or down regulations or up regulations, of the levels of odorifying molecules in urine and/or feces via non-sexual hormones dependent pathway.

The invention also encompasses deslorelin for use in improving attenuation or reduction of the urine and/or feces odor in a non-human animal.

Finally, the invention also generally relates to the use of deslorelin for the manufacturing of a medicament for
attenuating or reducing urine and/or feces odor in a non-human animal, wherein after administration to said non-human animal, the deslorelin plasma concentration of said non-human animal is higher than 10 pg/mL.

It has been shown that the use of deslorelin in a cat or in a dog, in a male or in a female, modulates, i.e. down regulates or up regulates, the levels of a molecule in urine, whereas at the same time, the levels of derivatives of that molecule are increased or decreased in urine.

It has been found that the use of deslorelin in a cat or in a dog, in a male or in a female, modulates, i.e. down regulates or up regulates the levels of a molecule in feces, whereas at the same time, the levels of derivatives of that molecule are increased or decreased in feces.

It has also been found that the use of deslorelin in a cat or in a dog, in a male or in a female, modulates, i.e. down regulates or up regulates, the levels of a molecule in urine, whereas at the same time, the levels of that same molecule or the levels of derivatives thereof in feces are increased or decreased.

It has also been found that the use of deslorelin in a cat or in a dog, in a male or in a female, modulates, i.e. down regulates or up regulates, the levels of a molecule in feces, whereas at the same time, the levels of that same molecule or the levels of derivatives thereof in urine are increased or decreased.

In addition, in feces several metabolomics molecules have also been identified. Indeed, from the data the following molecules have been selected from the following molecules: trimethylamine, 3-methyl butanoic acid and crotonic acid.

Indeed, cat feces had trimethylamine, a rotten fish odor that was not identified by GC-MS in fresh feces samples. However, a foul smell of fish was recorded by an odor specialist confirming the fact that a minute level of concentration can be detected by living sensory organs. Therefore, attenuation of the odor induced by trimethylamine is sought.

The present invention provides several advantages that includes but are not limited to the following:

First of all, with an action upon at least two synthesis pathways of urine or feces odor, it is clear that the results in terms of odor attenuation, will be at least the addition of these two pathways results and certainly better than action only based on urine/feces odor synthesis mediated by sexual hormone pathway alone.

In comparison with surgically neutered animal, it is now shown in chemically neutered animal by deslorelin administration that the molecules responsible of urine or feces odor are quantitatively and qualitatively altered; some are attenuated or reduced.

In addition, through the present disclosure it is demonstrated that the method of using deslorelin as described herein is a reliable, reversible, non-surgical neutering solution to foster pet family wellbeing while keeping options open.

The action of deslorelin through at least the testosterone pathway and particularly in reducing its blood level may contribute to the prevention of renal diseases in said animal because it is known these kind of diseases are related with the accumulation of molecules such as trimethylamine.

It should be also mentioned that further to a safety study in cat, deslorelin administration at high dose reveals neither serious adverse events nor live threatening events. In addition, according to several tolerance studies in male and female species, it has been concluded that deslorelin, particularly administrated as an implant (i.e. Superlorin®) was well tolerated.

### BRIEF DESCRIPTION OF DRAWINGS

In addition to the exemplary aspects and embodiments described above, further aspects and embodiments of the invention will become apparent by reference to the drawings and by study of the following descriptions.

FIG. 1 illustrates the synthesis of isovalthine and isobuteine in some Felidae species through the cooperation of liver and kidneys.

FIG. 2 illustrates the evolution of the serum testosterone concentration (in pg/mL) of male cats implanted with one 4.7 mg deslorelin implant (Suprelorin®) (continuous curve) or administered saline on D0 (discontinuous curve) (p<0.05) as described in example 1.

FIG. 3 illustrates the testosterone plasmatic levels of 4 male cats implanted with one 4.7 mg deslorelin implant (Suprelorin®) (FIG. 3A—each curve corresponds to one of the 4 cats) and of 3 male cats administered saline on D0 (FIG. 3B—each curve corresponds to one of the 3 cats) as described in example 2.

FIG. 4 is a graph presenting the mean relative abundance of felinine present in male cat urine samples collected at D63 after administration of saline (control group) or implanted with one 4.7 mg deslorelin implant (Suprelorin®) (chemically treated group) or in surgically castrated male cats (surgically castrated group) according to the protocol detailed in example 2.

FIG. 5 illustrates the mean relative abundance of MBCG present in male cat urine samples collected at D63 after administration of saline (control group) or implanted with one 4.7 mg deslorelin implant (Suprelorin®) (chemically treated group) (FIG. 5A) and of MBCG present in male cat urine samples collected at D63 after administration of saline (control group) and in surgically castrated male cats (surgically castrated group) (FIG. 5B) according to the protocol detailed in example 2.

FIG. 6 illustrates the mean relative abundance of N-acetylcadaverine present in male cat urine samples collected at D63 after administration of saline (control group) or implanted with one 4.7 mg deslorelin implant (Suprelorin®) (chemically treated group) (FIG. 6A) and of N-acetylcadaverine present in male cat urine samples collected at D63 after administration of saline (control group) and in surgically castrated male cats (surgically castrated group) according to the protocol detailed in example 3.

FIG. 7 illustrates the mean relative abundance of N-acetylputrescine present in male cat urine samples collected at D63 after administration of saline (control group) or implanted with one 4.7 mg deslorelin implant (Suprelorin®) (chemically treated group) according to the protocol detailed in example 3.

FIG. 8 illustrates the p-coumaroylputrescine urinary variation levels in a deslorelin treated cats group (Suprelorin®) (chemically treated group, discontinuous curve) vs a cats control group (continuous curve) according to the protocol of example 3.

FIG. 9 illustrates the mean relative abundance of taurine present in male cat urine samples collected at D63 after administration of saline (control group) or implanted with one 4.7 mg deslorelin implant (Suprelorin®) (chemically treated group) according to the protocol detailed in example 3.

FIG. 10 illustrates the mean relative abundance of valine present in male cat urine samples collected at D42 after administration of saline (control group) or implanted with one 4.7 mg deslorelin implant (Suprelorin®) (chemically treated group) (FIG. 10A) and of valine present in male cat urine samples collected at D42 after administration of saline (control group) and in surgically castrated male cats (surgically castrated group) (FIG. 10B) according to the protocol detailed in example 3.

FIG. 11 is a graph representing the mean relative abundance of indole present in male cat urine samples collected at D63 after administration of saline (control group) or implanted with one 4.7 mg deslorelin implant (Suprelorin®) (chemically treated group) (FIG. 11A) and of indole present in male cat urine samples collected at D63 after administration of saline (control group) and in surgically castrated male cats (surgically castrated group) (FIG. 11B) according to the protocol detailed in example 3.

FIG. 12 illustrates the mean relative abundance of isovalthine present in male cat urine samples collected at D63 after administration of saline (control group) or implanted with one 4.7 mg deslorelin implant (Suprelorin®) (chemically treated group) according to the protocol detailed in example 3.

FIG. 13 is a graph representing the mean relative abundance of mercaptopyruvic acid present in male cat urine samples collected at D63 after administration of saline (control group) or implanted with one 4.7 mg deslorelin implant (Suprelorin®) (chemically treated group) (FIG. 13A) and of mercaptopyruvic acid present in male cat urine samples collected at D63 after administration of saline (control group) and in surgically castrated male cats (surgically castrated group) (FIG. 13B) according to the protocol detailed in example 3.

FIG. 14 illustrates the mean relative abundance of 3-mercaptopropionic acid present in male cat urine samples collected at D63 after administration of saline (control group) or implanted with one 4.7 mg deslorelin implant (Suprelorin®) (chemically treated group) according to the protocol detailed in example 3.

FIG. 15 is a graph representing the mean relative abundance of 7-mercapto heptanoylthreonine present in male cat urine samples collected at D63 after administration of saline (control group) or implanted with one 4.7 mg deslorelin implant (Suprelorin®) (chemically treated group) (FIG. 15A) and of 7-mercapto heptanoylthreonine present in male cat urine samples collected at D63 after administration of saline (control group) and in surgically castrated male cats (surgically castrated group) (FIG. 15B) according to the protocol detailed in example 3.

FIG. 16 illustrates the mean relative abundance of 2-mercapto-2-methylpentan-4-one present in male cat urine samples collected at D63 after administration of saline (control group) or implanted with one 4.7 mg deslorelin implant (Suprelorin®) (chemically treated group) according to the protocol detailed in example 3.

FIG. 17 illustrates the mean relative abundance of crotonic acid present in male cat feces samples collected at D63 after administration of saline (control group) or implanted with one 4.7 mg deslorelin implant (Suprelorin®) (chemically treated group) according to the protocol detailed in example 3.

FIG. 18 is a graph representing the mean % of efficacy of administration of Suprelorin 4.7 mg in male cats for studies A and b of example 4 at different times of observation

FIGS. 19A and 19B report the detailed levels of Testosterone concentration (in pg/mL) for study 1 (FIG. 19A) and for study 2 (FIG. 19B) according to example 4

FIGS. 20A and 20B present respectively the mean of sum score of sexual behaviors for study 1 (FIG. 20A) and for study 2 (FIG. 20B) for study 2 of example 4

FIGS. 21A and 21B show respectively the mean vocalization variation for study 1 (FIG. 21A) and for study 2 (FIG. 21B) of example 4

FIGS. 22A and 22B show respectively the urine marking, the % change from baseline is respectively for study 1 (FIG. 22A) and for study 2 (FIG. 22B) of example 4

FIGS. 23A and 23B show the urine odor variation/reduction measured as the change from baseline respectively for study 1 (FIG. 23A) and for study 2 (FIG. 23B) of example 4.

FIGS. 24A and 24B show the % change of testicular volume respectively for study 1 (FIG. 24A) and for study 2 (FIG. 24B) of example 4.

FIGS. 25A and 25B show the % change of appearance penile spines respectively for study 1 in (FIG. 25A) and for study 2 (FIG. 25B) of example 4.

### DETAILED DESCRIPTION OF INVENTION

One of the purpose of the present application is to show that deslorelin administration (for example when administered in an implant such as Suprelorin®) decreases urine and/or feces odor, in particular unpleasant urine and/or feces odors. Surprisingly, it has been shown by the inventors that odor reduction is mediated via at least two pathways. Indeed, one such pathways is the “classic” testosterone-mediated sex hormone pathway. The inventors have also demonstrated at least another pathway unrelated to sex hormones as described in the present application.

Hence, the objective of decreasing the odor of urine and/or feces in an animal is solved by using deslorelin which attenuates or reduces urine and/or feces unpleasant odor in inducing a chemical castration of administrated non-human animal.

Indeed, Inventors have shown that deslorelin administration has an impact on different molecules involved in urine and/or feces excretion. According to the degradation or synthesis of those molecules, they are also responsible of urine or feces odor. Therefore is it one object of the invention to intervene with the use of deslorelin in the composition of urine and/or feces odor by modifying or altering and particularly in down regulating or up regulating the concentration of particular molecules in urine and/or feces. Examples of deslorelin action are developed further below.

The term “modulating” refers herein to a modification in the quantity of a molecule in response to deslorelin administration. Modulation includes both upregulation and downregulation.

The term “downregulating” refers herein to the reduction in quantity of a molecule in response to deslorelin administration comparatively to the quantity of said molecule in urine and/or feces before the start of deslorelin administration.

The term “upregulating” refers herein to the increase of the quantity of a molecule in response to deslorelin administration comparatively to the quantity of said molecule in urine and/or feces before the start of deslorelin administration.

The terms “continuously” and substantially continuously” in reference to the administration of deslorelin means that deslorelin is administered without interruption or substantially without interruption for example via implantation of a sustained release drug delivery system.

Said molecules can be measured by methods known by skilled person in the art, which includes tandem mass spectrometry (also known as MS/MS or MS2), two-dimensional gas chromatography mass spectrometry (also known as GC×GC-MS method and as described for example by C. Suzuki et al., Journal of Chemical Ecology, 45, 579-587 (2019)) and Liquid Chromatography-MS/MS.

According to a preferred embodiment of the present invention, deslorelin down regulates the urinary concentration of molecules selected in the group comprising precursors such as 3-paracresol, felinine, felinine methylbutanolcysteinylglycine (MBCG), felinine derivatives such as N-acetyl felinine, and 3-mercapto-3-methylbutan-1-ol, isovalthine, isobuteine, indole, cadaverine and derivatives thereof such as N-acetylcadaverine, putrescine and derivatives thereof such as N-acetylputrescine and p-coumaroylputrescine, mercapto compounds such as mercaptopyruvic acid, 3-mercaptopropionic acid, 7-mercaptoheptanoylthreonine, and taurine.

According to a most preferred embodiment, deslorelin downregulates the urinary concentration of molecules selected in the group comprising felinine and felinine precursors thereof such as 3-methylbutanolcysteinylglycine, indole, cadaverine and derivates thereof such as N-acetylcadaverine, putrescine and derivates thereof such as N-acetylputrescine and p-coumaroylputrescine, mercaptopyruvic acid, 3-mercaptopropionic acid, 7-mercaptoheptanoylthreonine and taurine.

According to another preferred embodiment of the present invention, deslorelin upregulates the urinary concentration of molecules selected in the group comprising 2-mercapto 2-methylpentan-4-one (rather pleasant onion key food odorant) and valine (pleasant smell of vanilla and maple syrup).

According to a most preferred embodiment of the present invention, deslorelin upregulates the urinary concentration valine.

According to another preferred embodiment of the present invention, deslorelin down regulates the feces concentration of molecules selected from the group comprising crotonic acid and trimethylamine.

As it will be explained hereafter the downregulation and upregulation of these different molecules may be influenced by two distinct synthesis pathways.

### Urine or Feces Odor Synthesis Pathway Via Sexual Hormones

It is known from prior art that testosterone increases the free excretion of felinine, N-acetylfelinine and 3-methylbutanolglutathione in neutered adult male cats and intact females, whereas estradiol does not modulate this effect (Hendriks W H et al., Testosterone increases urinary free felinine, N-acetylfelinine and methylbutanolglutathione excretion in cats (Felis catus)., J Anim Physiol Anim Nutr (Berl)., 2008 February; 92 (1): 53-62).

Felinine and N-acetylfelinine were also detected in various tissues. Furthermore all compounds, were detected in cat bile and fecal samples, indicating that felinine is excreted into the feces via bile from the liver (LC-MS/MS quantification of felinine metabolites in tissues, fluids, and excretions from the domestic cat (Felis catus), Futsuta et al, J Chromatogr B Analyt Technol Biomed Life Sci., 2018 Jan. 1; 1072:94-99).

Thus, the decrease of felinine levels and/or its metabolites levels and other molecules will impact the urine or feces odor.

It is also known from prior art (M. Miyazaki et al., Comp Biochem Physiol B Biochem Mol Biol, 2006 November-December; 145 (3-4): 270-7) that cauxin excretion is sex-dependent. In mature cats, cauxin excretion was higher in intact males than in castrated males or in intact or spayed females. Daily cauxin excretion decreased immediately after castration. Immunohistochemistry confirmed that cauxin expression in the kidney proximal straight tubules was higher in intact males than in castrated males.

These results suggest that the cauxin excretion is regulated by sex hormones, such as testosterone and that the decomposition products by cauxin are excreted in a species-, sex-, and age-dependent manner, as is cauxin itself.

In addition, cauxin, a carboxylesterase excreted as a major urinary component, regulates felinine production. In vitro enzyme assays indicated that cauxin hydrolyzed the felinine precursor 3-methylbutanolcysteinylglycine to felinine and glycine.

Surprisingly the inventors of the present invention have found and demonstrated that the chemical castration induced by deslorelin release impacts the cauxin as well as the felinine production. Felinine is also responsible of urine odor as well as derivative molecules like N-acetyl felinine, 3-methylbutanolcysteinylglycine (MBCG) and 3-mercapto-3-methylbutan-1-ol.

It is one object of the present invention to reduce or attenuate urine odor via the urine or feces odor synthesis pathway via sexual hormones by using deslorelin in a non-human animal, wherein after administration to said non-human animal, the deslorelin plasma concentration of said non-human animal is higher than 10 pg/mL.

Therefore, according to a first object, the invention related to deslorelin for use in attenuating or reducing urine and/or feces odor in a non-human animal, wherein after administration to said non-human animal, the deslorelin plasma concentration of said non-human animal is higher than 10 pg/mL.

More specifically, after the administration to said non-human animal, the deslorelin plasma concentration is between 10 pg/mL and 400 pg/ml, from 8 days after administration up to at least 6 months. Preferentially, deslorelin is administered or released after administration continuously in said non-human animal.

A second object of the invention is also a process for attenuating or reducing urine and/or feces odor in a non-human animal, the process consisting in administering to the non-human animal a quantity of deslorelin wherein after administration to said non-human animal, the deslorelin plasma concentration of said non-human animal is higher than 10 pg/mL.

According to another embodiment, the invention relates to a process for attenuating or reducing urine and/or feces odor in a non-human animal, the process consisting in administering to the non-human animal a quantity of deslorelin that will induce modulations, i.e. down regulations or up regulations of the levels of odorifying molecules in urine and/or feces.

Preferably according to this embodiment, the non-human animal is an intact male animal, preferably a canine or feline and even more preferably a dog or a cat, and most preferably a cat.

Preferably, according to this embodiment, deslorelin induces modulations or down regulations or up regulations of the levels of odorifying molecules in urine and/or feces via sex hormone synthesis pathway.

As it has been demonstrated in the examples illustrating the present invention, that the concentration of felinine and felinine precursors such as 3-methylbutanolcysteinylglycine is decreased after the administration of deslorelin in intact male adult cats.

Therefore, according to a particularly preferred embodiment, the invention relates to deslorelin for use in downregulating the concentration of felinine and felinine derivatives in urine in a non-human animal.

### Urine or Feces Odor Synthesis Via Non-Sexual Hormones Dependent Pathway

In addition to the classic sex hormone pathway mediated by testosterone responsible for the odor of urine and/or feces, the inventors have also discovered another pathway unrelated to sex-dependent hormones. Indeed, as shown in the examples, the inventors have identified new molecules as being molecules responsible of urine and or feces odor. These molecules are secreted outside the sexual hormone pathway. In fact, those molecules are not derivate from Testosterone cascade.

Therefore, one aspect of the present invention is the use of deslorelin to modify or alter or to down regulate or up regulate the levels of molecules selected from the group consisting of crotonic acid; indole; paracresol; cadaverine and cadaverine derivatives such as acetylcadaverine, and glutathionylaminopropylcadaverine; putrescine and putrescine derivatives such as N-acetylputrescine, and p-coumaroylputrescine; mercapto derivatives selected from the group comprising 2-mercapto-3-butanone, 2-mercaptoethanol, 3-mercaptohexyl butyrate, mercaptopyruvic acid, 3-mercaptopropionic acid, 3-mercapto-3-methylbutan-1-ol, 7-mercaptoheptanoylthreonine, 1alpha,5alpha-mimercaptoandrostane-3alpha,17beta-diol, 2-mercapto-2-methylpentan-4-one, taurine, valine, isovalthine, isobuteine and trimethylamine.

According to a preferred embodiment, deslorelin down regulates the urinary levels of molecules selected from the group comprising isovalthine, isobuteine, indole, cadaverine and derivatives thereof such as N-acetylcadaverine, putrescine and derivatives thereof such as N-acetylputrescine and p-coumaroylputrescine, mercapto compounds such as mercaptopyruvic acid, 3-mercaptopropionic acid, and 7-mercaptoheptanoylthreonine and taurine.

According to a most preferred embodiment, deslorelin down regulates the levels of molecules selected from the group comprising indole, cadaverine and derivates thereof such as N-acetylcadaverine, putrescine and derivatives thereof such as N-acetylputrescine and p-coumaroylputrescine, and mercaptopyruvic acid.

According to another preferred embodiment, deslorelin upregulates the urinary levels of molecules selected from the group comprising 2-mercapto 2-methylpentan-4-one and valine.

According to another most preferred embodiment, deslorelin up regulates the level of valine.

Isovalthine is a branched-chain sulfur amino acid, which has been found in the urine of normal cats. Isovalthine concentrations in the urine of healthy adult cats are approximately 24-66 micromol/l and are not affected by the sex of the cat.

Isovalthinuria can be induced in other species (rats, rabbits, guinea pigs, humans, dogs) following the administration of certain inducing agents such as certain cholesterol-lowering agents, bile acids, hormones or cholesterol precursors. The method of induction of isovalthinuria was widely studied during the 1960s and efforts were made to understand its biosynthesis.

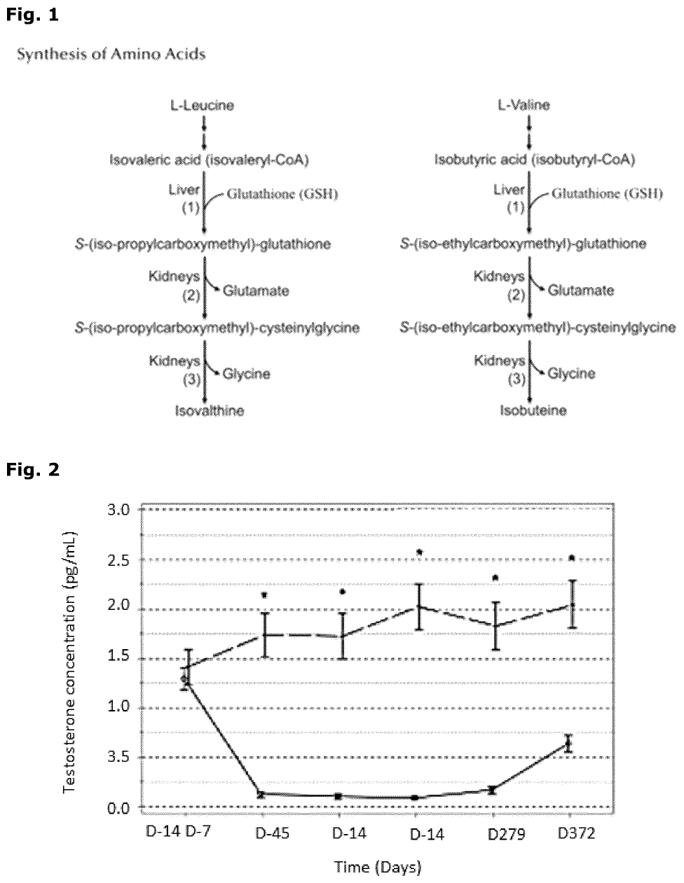

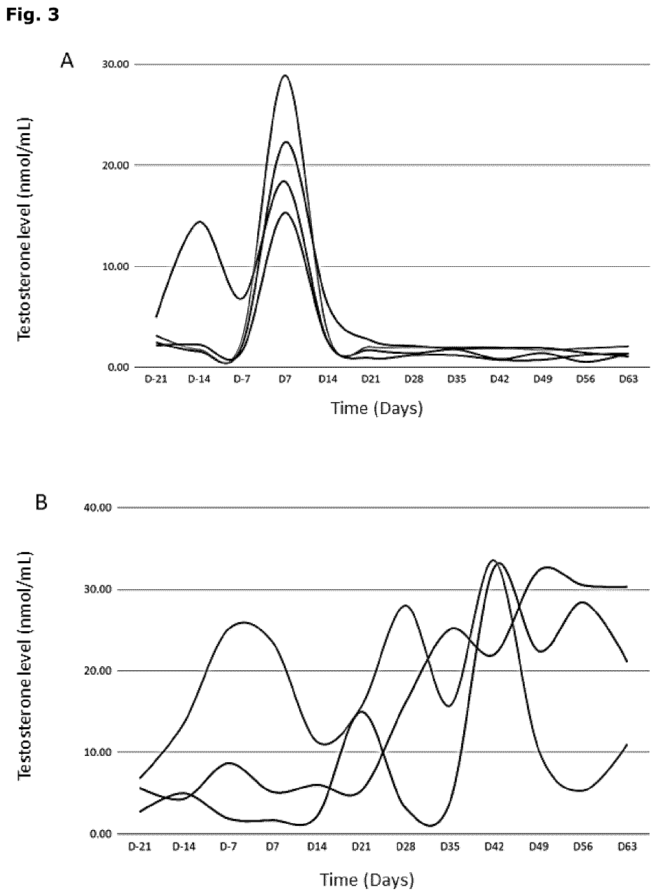

However, although the origin of the sulfur atom in isovalthin has been shown to be from cysteine or methionine as shown in FIG. 1, the origin of the carbon skeleton remains unknown. Interest in isovalthin metabolism has been generated in part because it is believed to have been found in the urine of patients with hypercholesterolemia. Thus little is known about isovalthin.

Inventors now have found that isovalthin is one molecule responsible of urine odor. As it is known that urine is a mix of several molecules, it is considered that isovalthin is one of the molecules which has an active part in odor release.

Little is also know about isobuteine, also described as 2-amino-6-carboxy-4-thiaheptanoic acid. This amino acid has been isolated from the urine of healthy humans and cats, but the biological role of isobutein is also unknown.

Inventors now have found that isobutein is one molecule responsible of urine odor. As it is known that urine is a mix of several molecules, it is considered that isobutein is one of the molecules which has an active part in odor release.

In addition, in feces several metabolomics molecules have also been identified. Indeed, from the data the following molecules have been identified: trimethylamine and 3-methyl butanoic acid.

Hence, with chemical castration induced by deslorelin administration, the urine and/or feces odor is also alleviated in the same magnitude of time. The inventors have surprisingly found that with chemical castration with deslorelin, the urine odor is attenuated after several weeks. Generally, the odor is alleviated between 21 and 42 days; meaning approximately between 3 and 6 weeks.

In one aspect of the invention, the odor of urine in unsterilized animals, particularly in domestic male cats, is reduced by administering deslorelin to the animal.

It is also noticed that some molecules are down-regulated in urine but upregulated in feces or inversely. This shows a compensation mechanism between molecules levels elimination in urine and feces at different levels in comparison with non-chemically castrated animals (data not shown).

Therefore, the invention also relates to a process for improving the attenuation or reduction of the urine and/or feces odor in a non-human intact male animal compared to a neutered male, the process consisting in administering to the non-human intact male animal a quantity of deslorelin that will induce modulations or down regulations or up regulations of the levels of odorifying molecules in urine and/or feces.

According to this embodiment, deslorelin will induce modulations or down regulations or up regulations of the levels of odorifying molecules in urine and/or feces via non-sexual hormones dependent pathway.

According to another embodiment, the invention relates to deslorelin for use in improving attenuation or reduction of the urine and/or feces odor in a non-human animal.

### Example 1: Large Scale Field Evaluation of the Efficacy and Safety of a Single 4.7 mg Deslorelin Implant to Suppress Testosterone, Sexual Behaviors and Urine Odour in Male Cats

The aim of this multi-site field, double-masked, placebo-controlled, randomized study was to confirm that deslorelin implants can be used as a neutering option for male cats and can also reduce their associated tomcat sexual behaviours.

### 1.1. Materials and Methods

Privately owned indoor male cats [3 months-11 years of age] were allocated to two groups to be either implanted with one 4.7 mg deslorelin implant (Suprelorin® 4.7 mg, Virbac, France) or to receive sodium chloride. The implant and the saline solution were administered subcutaneously between the shoulder blades on day 0 (D0). Serum testosterone concentrations in ng/ml (T), sexual behaviors (presence of urine marking, frequency of vocalization, frequency of aggressiveness) and presence of intact male cat urine odour were assessed until month (M) 12 (D372). General safety assessments, bodyweight, appetite and adverse reactions were regularly followed.

The protocol received ethical approval. The Cochran-Mantel-Haenszel test stratified by center with the RIDIT transformation and the general association statistic were used for comparison between groups.

Among n=205 Male Cats, n=154 received Suprelorin® 4.7 mg (treated group) and n=51 received the saline solution as control group.

### 1.2 Results

As shown in FIG. 1, in the treated group, mean T values decreased and were significantly lower compared to control by D45 (0.13±0.375 vs 1.74±1.547 ng/ml) until M12 (0.64±1.138 vs 2.04±1.708 ng/ml).

Sexual behavior scores, testicular volumes, visibility of penile spines and urine odour also decreased significantly, and were significantly lower in the deslorelin group relative to the saline group throughout the study. No flare in sexual behavior and no safety concerns were observed.

### 1.3 Conclusion

This study shows that Suprelorin® 4.7 mg implants can be used in male cats from 3 months of age as an effective and safe neutering option for at least one year with the associated reduction of tomcat sexual behaviours and reduction of urine odour.

### Example 2: Evaluation of Alteration of Molecules Levels Linked to Sexual Hormone Dependent Pathway

The aim of these studies was to quantify the levels of certain molecules responsible for urine odor. It was also an objective of this study to evaluate the plasmatic level of testosterone and the metabolomics parameters present in urine in chemically castrated male cat with deslorelin as well as in surgically castrated male cats. Hence, several parameters were assessed. In particular dosages have been performed in Serum/plasma and Urine.

### 2.1 Materials and Methods

A first study A was performed in 2 groups of a total number of 7 male healthy cats old from 12 to 13 months and weighting between 4 and 6 kg in:

Control group: n=3, intact males, administered sodium chloride (saline) on day 0 (D0),

Chemically treated group: n=4 one implant of Suprelorin® 4.7 mg on day 0 (D0).

The study A was conducted for a period of 63 days after the implantation of Suprelorin® 4.7 mg.

From D-21 to D-7, a clinical examination of the cats was performed (body weight, temperature) and samples of urine and feces were collected. At D0, the cats of the treated group were implanted subcutaneously with Suprelorin® 4.7 mg (Virbac, France).

From D7 to D63, a weekly clinical examination of the cats was performed (body weight, temperature) and samples urine and feces were collected.

A non-classical analyzing technic based on tandem mass spectrometry, also known as MS/MS or MS2 was used to analyze the samples and the search of molecules responsible of odor in urine and feces samples. Thanks to this unusual technic, the metabolomics present in samples have been assessed.

The plasmatic levels of testosterone has also been evaluated for the chemically treated group and the control group.

Metabolomic analysis were performed on urine and feces samples collected prior the treatment (at D-14), enabling to compare the groups and confirm the same levels of molecules observed among the two groups (which was confirmed, data not shown).

Metabolomic analysis were also performed after treatment at D49 for feces and D63 for urines in order to compare the molecules levels between treated animals and control animals to evaluate the effect of the castration on the levels of odoring molecules.

A second study B was performed in 2 groups of a total number of 4 male healthy cats old from 12 to 13 months and weighting between 4 and 6 kg:

Control group: n=2, intact males, administered sodium chloride (saline) on day 0 (D0),

Surgically castrated group: n=4 (surgical castration by removal of testicles; Adult cats neutered for more than 6 months.), administered sodium chloride (saline) on day 0 (D0)

The protocol was the same as in the first study A.

### LC-MS/MS Analysis

Metabolomic analysis were performed using LC-MS/MS.

Liquid chromatographic analysis was performed using the DIONEX Ultimate 3000 HPLC System (Thermo Fisher Scientific). 10 μL of each sample was injected into a Synergi 4 μm Hydro-RP 80 Å, 250×3.0 mm column (Phenomenex, Le Pecq, France). The mobile phases were composed of 0.1% formic acid (Thermo Fisher Scientific) in water (Study A) and 0.1% formic acid in acetonitrile (Study B). The gradient was set as follows with a flow rate of 0.9 mL/min: 0% phase B from 0 to 5 min, 0-95% B from 5 to 21 min, holding at 95% B to 21.5 min, 95-0% B from 21.5 to 22 min, holding at 0% B until 25 min for column equilibration. Mass spectrometry Mass spectrometry analysis was carried out on a Q Exactive Plus Orbitrap mass spectrometer (Thermo Fisher Scientific) with a heated electrospray ionization source, HESI II, operating in positive/negative ion-switching mode. Additional analyses of the samples were operated in positive mode. High-resolution accuratemass full-scan MS and top 5 MS2 spectra were collected in a data-dependent fashion at a resolving power of 70,000 and 35,000 at m/z 400, respectively. All samples were successively treated in the same run.

### Metabolomic Analysis

Post treatment of the data was performed using Compound Discovers 3.2 (Thermo Fisher Scientific).

The workflow template was untargeted metabolomics with statistics to detect unknowns with ID using online Databases and mzLogic.ons obtained from positive and negative ionization mode using MZmine (Version 2.39) were also analyzed.

Mass detection was performed using the Mass detector tool (mass detector: Wavelet transform, MS level 1; Noise level 10\hat{ }5, scale level: 5, Wavelet window size: 30%). Chromatograms were detected using the ADAP chromatogram builder (MS level: 1, Minimum group size in number of scans: 5, Group intensity threshold: 5×10\hat{ }2, minimum highest intensity: 10\hat{ }5, m/z tolerance: 10 ppm). Peaks were separated using the Peak extender module (M/Z tolerance: 10 ppm, minimum height: 10\hat{ }5). Retention times were normalized using the retention time calibration module (m/z tolerance: 10 ppm; retention time tolerance (relative): 10%, minimum height: 10\hat{ }5). Peaks were then aligned using the RANSAC aligner (random sample consensus) algorithm with a tolerance of 10 ppm in m/z and 1 min in retention time. Peaks were then identified using the Human metabolome database (HMDB, version 3.0) with 10 ppm of mass tolerance. Missing values were filled in using the same m/z and RT range gap filler with a tolerance of 10 ppm in m/z. The results obtained with each polarity were combined and, for metabolites that were identified in both modes, only the mode for which the peak had the highest mean intensity was considered.

### 2.2 Results

### Felinine:

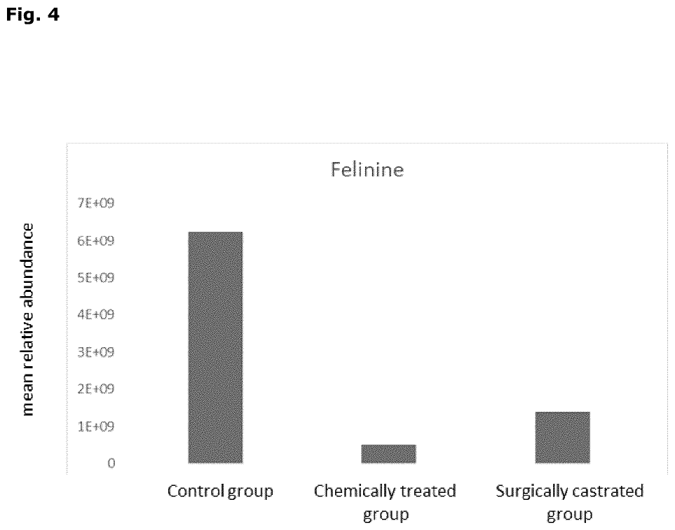

The results are reported on FIG. 4 annexed. On this figure, the mean relative abundance peak of felinine present in male cat urine samples collected at D63 after administration of saline (control group) is compared with that of cats implanted with one 4.7 mg deslorelin implant (Suprelorin®) (chemically treated group) and with that of surgically castrated male cats (surgically castrated group).

The mean relative abundance peak of felinine in both control groups from Study A and study B being identical, the results are shown on a combined graph (FIG. 4).

These results show that on Day 63, felinine was significantly decreased in the both chemically treated and surgically castrated group versus the control group.

### MBCG:

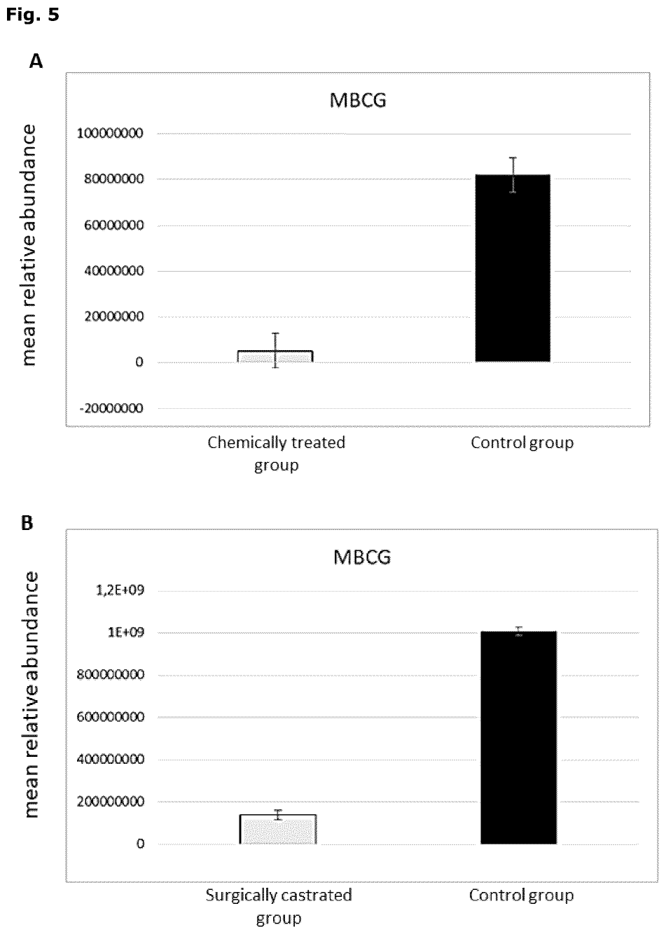

The results are reported on FIGS. 5A and 5B annexed. On FIG. 5A, the mean relative abundance of MBCG present in male cat urine samples collected at D63 after administration of saline (control group) is compared with that of cats implanted with one 4.7 mg deslorelin implant (Suprelorin®) (chemically treated group). On FIG. 5B, the mean relative abundance of MBCG present in male cat urine samples collected at D63 after administration of saline (control group) is compared with that of surgically castrated male cats (surgically castrated group).

These results show that on Day 63, MBCG was significantly decreased in the both chemically treated and surgically castrated group versus the control group.

### Example 3: Evaluation of Alteration of Molecules Levels Linked to Non-Sexual Hormone Dependent Pathway

The aim of this study was to evaluate whether the use of deslorelin can modify or alter or down regulate or up regulate the levels of molecules identified as being responsible of urine odor.

The aim of this study was also to demonstrate the use deslorelin can also modify the level of crotonic acid in the feces which is one of the molecules responsible of feces odor.

### 3.1 Material and Methods

The studies are the same performed according to the protocol in example 2 with same groups of cats.

A non-classical analyzing technic based on tandem mass spectrometry, also known as MS/MS or MS2 was used to analyze the samples and the search of molecules responsible of odor in urine and feces samples as already detailed in Example 2. Thanks to this unusual technic, the metabolomics present in samples have been assessed:

The levels of the following metabolomics were measured in urine collected samples of each group during the study: N-acetylcadaverine, N-acetylputrescine, p-coumaroylputrescine, taurine, valine, indole, isovalthine, and mercapto derivatives, namely mercaptopyruvic acid, mercaptopropionic acid, 3-mercaptoheptanoyl threonine, and 2-mercapto-2-methylpentan-4-one, which are all linked to non-sexual hormone dependent pathway. These molecules have been identified as being one of the molecules responsible of urine odor.

The levels of crotonic acid were measured in the feces collected samples of each group during the study.

Tandem mass spectrometry (MS/MS or MS2) performs mass analysis at least twice while performing a dissociation process or chemical reaction to change the mass or charge of an ion. MS/MS improves specificity beyond separation by molecular weight and provides insight into structure elucidation and identifying elementary composition. The most common dissociation method is collision-Induced dissociation (CID), in which the first analyzer is used to isolate a specified target ion (precursor ion) and, subsequently, undergoes a fragmentation process to yield neutral fragments and fragment ions (product ions). The second analyzer analyzes these product ions and forms a unique pattern based on the precursor ion structure and components. (Richard A. McPherson, Methods in Enzymology, 2019).

MS/MS is especially useful for analyzing complex mixtures and involves two stages of MS. In the first stage of MS/MS, a predetermined set of m/z ions are isolated from the rest of the ions coming from the ion source and fragmented by a chemical reaction. In the second stage, mass spectra are produced for the fragments. TANDEM MS is generally used for bioanalysis of drugs. Identification and determination of phase I and phase II drug metabolites are achieved by TANDEM MS coupled with HPLC (Glish, G. L. and Vachet, R. W., 2003. Nature Reviews Drug Discovery 2 (2): 140; Holčapek, M. et al., 2008., Analytical and Bioanalytical Chemistry 391 (1): 59-78).

### 3.2 Study Results

### N-acetylcadaverine

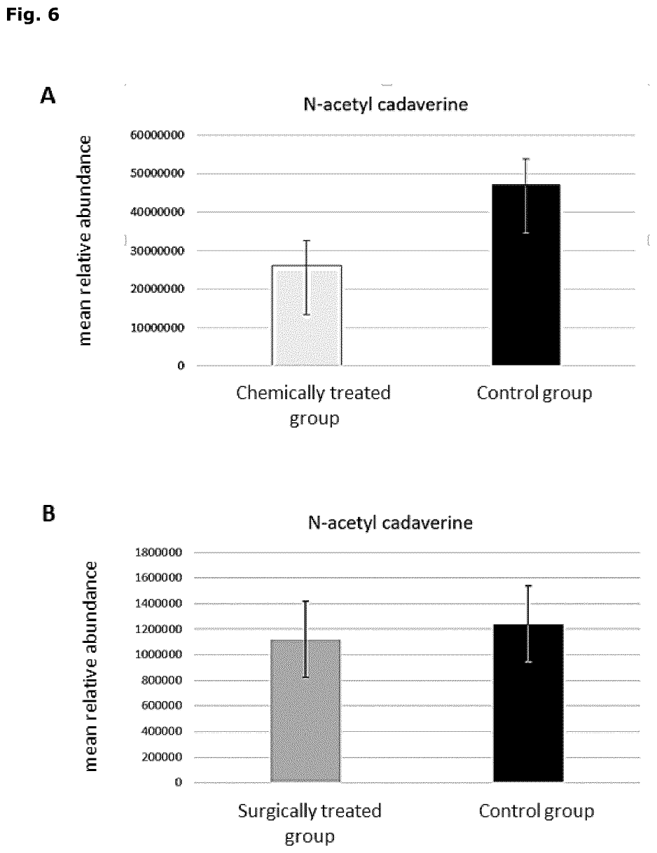

The results are reported on FIG. 6 annexed. On this figure, the mean relative abundance of N-acetylcadaverine present in male cat urine samples collected at D63 after administration of saline (control group) is compared with that of cats implanted with one 4.7 mg deslorelin implant (Suprelorin®) (chemically treated group) (FIG. 6A) and with that of surgically castrated male cats (surgically castrated group) (FIG. 6B).

These results show that on Day 63, N-acetylcadaverine, known to have a foul odour of putrefying flesh, was significantly decreased in the chemically treated group versus the control group while on the contrary there is no significantly difference between the N-acetylcadaverine concentration measured in the urinary samples of the surgically castrated group versus the control group.

### N-acetylputrescine

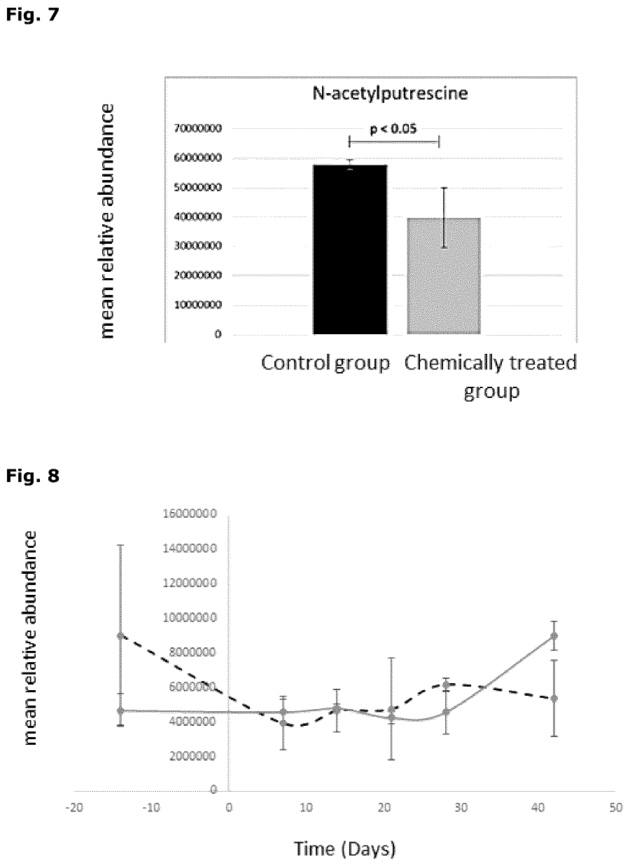

The results are reported on FIG. 7 annexed. On this figure, the mean relative abundance of N-acetylputrescine present in male cat urine samples collected at D63 after administration of saline (control group) is compared with that of cats implanted with one 4.7 mg deslorelin implant (Suprelorin®) (chemically treated group).

The results demonstrate that on Day 63, N-acetylputrescine, known to have a foul odour of putrefying flesh, was significantly decreased in the chemically treated group according to the invention versus the control group.

### p-coumaroylputrescine

Results are reported on FIG. 8 annexed. On this figure, the evolution of the mean relative abundance of p-coumaroylputrescine is expressed as a function of time (in days) for the control group (continuous curve) and for the chemically treated group (discontinuous curve).

These results the administration of deslorelin induces a decrease of the p-coumaroylputrescine concentration (chemically treated group) vs control group.

### Taurine:

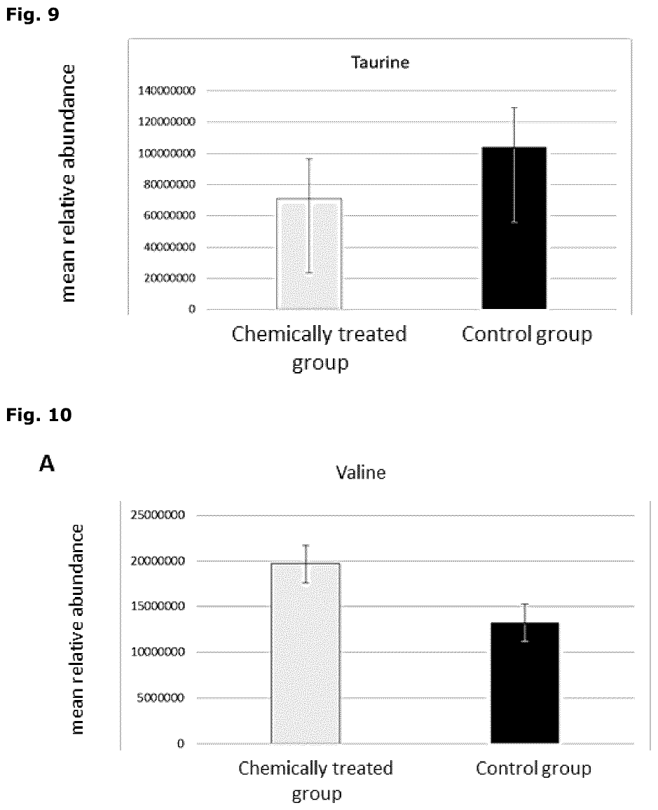

The results are reported on FIG. 9 annexed. On this figure, the mean relative abundance of taurine present in male cat urine samples collected at D63 after administration of saline (control group) is compared with that of cats implanted with one 4.7 mg deslorelin implant (Suprelorin®) (chemically treated group).

The results demonstrate that on Day 63, taurine, was significantly decreased in the chemically treated group according to the invention versus the control group.

### Valine:

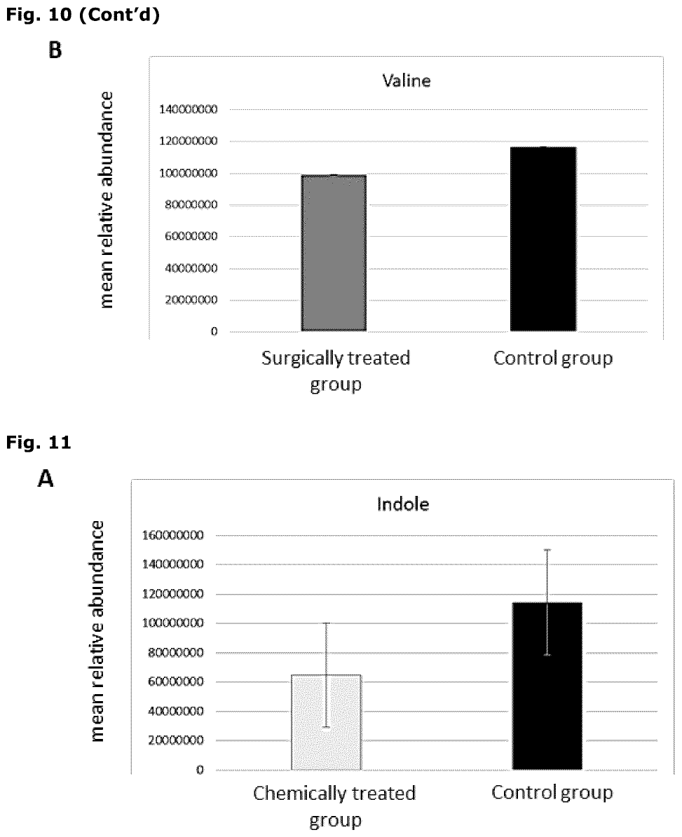

The results are reported on FIG. 10 annexed. On this figure, the mean relative abundance of valine present in male cat urine samples collected at D42 after administration of saline (control group) is compared with that of cats implanted with one 4.7 mg deslorelin implant (Suprelorin®) (chemically treated group) (FIG. 10A) and with that of surgically castrated male cats (surgically castrated group) (FIG. 10B).

These results show that on Day 42, valine, known to have a pleasant sweet odour, was significantly increased in the chemically treated group versus the control group while on the contrary there is no significantly difference between the valine concentration measured in the urinary samples of the surgically castrated group versus the control group.

### Indole:

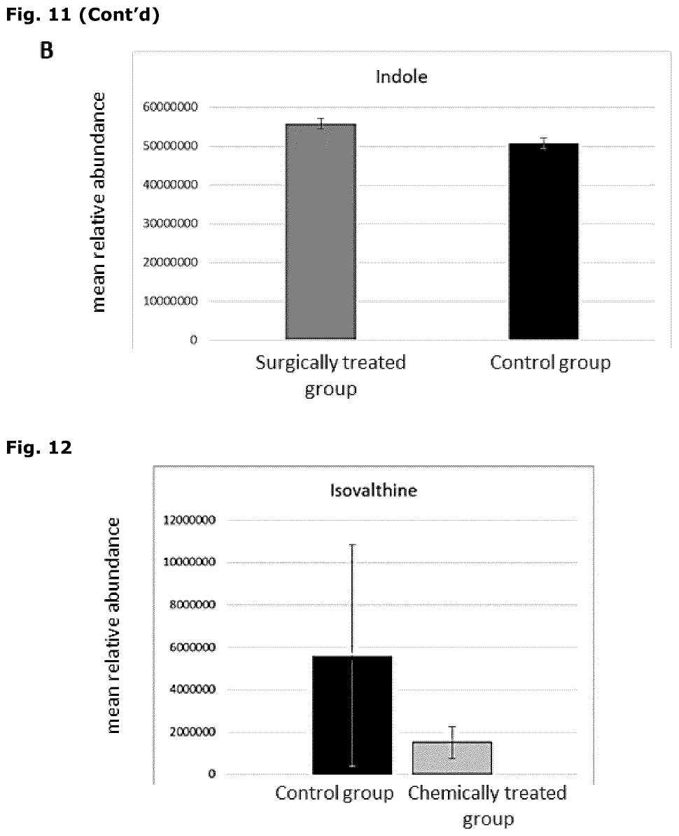

The results are reported on FIG. 11 annexed. On this figure, the mean relative abundance of indole present in male cat urine samples collected at D63 after administration of saline (control group) is compared with that of cats implanted with one 4.7 mg deslorelin implant (Suprelorin®) (chemically treated group) (FIG. 11A) and with that of surgically castrated male cats (surgically castrated group) (FIG. 11B).

These results show that on Day 63, indole, known to have an intense faecal odour, was significantly decreased in the chemically treated group versus the control group while on the contrary there is no significantly difference between the indole concentration measured in the urinary samples of the surgically castrated group versus the control group.

### Isovalthine:

The results are reported on FIG. 12 annexed. On this figure, the mean relative abundance of isovalthine present in male cat urine samples collected at D63 after administration of saline (control group) is compared with that of cats implanted with one 4.7 mg deslorelin implant (Suprelorin®) (chemically treated group).

The results demonstrate that on Day 63, isovalthine, known to have a foot/cheese odour, was decreased in the chemically treated group according to the invention versus the control group.

### Mercaptopyruvic Acid:

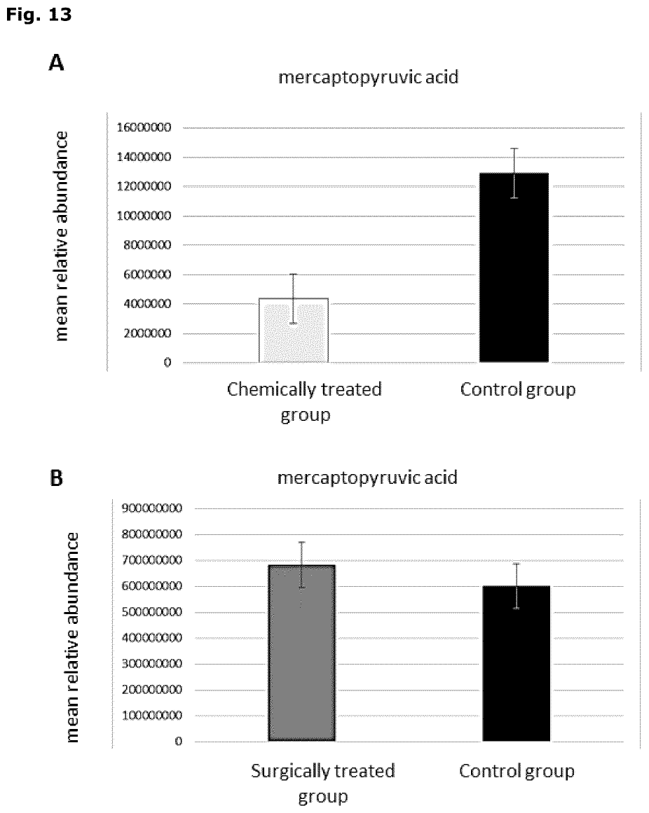

The results are reported on FIG. 13 annexed. On this figure, the mean relative abundance of mercaptopyruvic acid present in male cat urine samples collected at D63 after administration of saline (control group) is compared with that of cats implanted with one 4.7 mg deslorelin implant (Suprelorin®) (chemically treated group) (FIG. 13A) and with that of surgically castrated male cats (surgically castrated group) (FIG. 13B).

These results show that on Day 63, mercaptopyruvic acid, known as a stench compound, was significantly decreased in the chemically treated group versus the control group while on the contrary there is no significantly difference between the mercaptopyruvic acid concentration measured in the urinary samples of the surgically castrated group versus the control group.

### 3-mercaptopropionic Acid

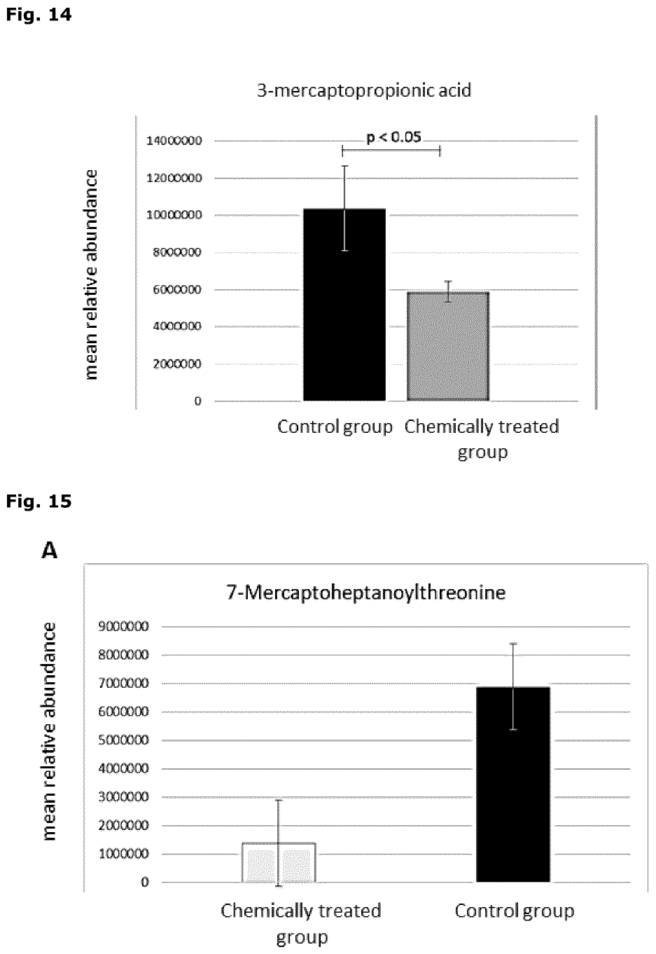

The results are reported on FIG. 14 annexed. On this figure, the mean relative abundance of 3-mercaptopropionic acid present in male cat urine samples collected at D63 after administration of saline (control group) is compared with that of cats implanted with one 4.7 mg deslorelin implant (Suprelorin®) (chemically treated group).

The results demonstrate that on Day 63, 3-mercaptopropionic acid, known as a stench compound, was significantly decreased in the chemically treated group according to the invention versus the control group.

### 7-mercapto heptanoylthreonine

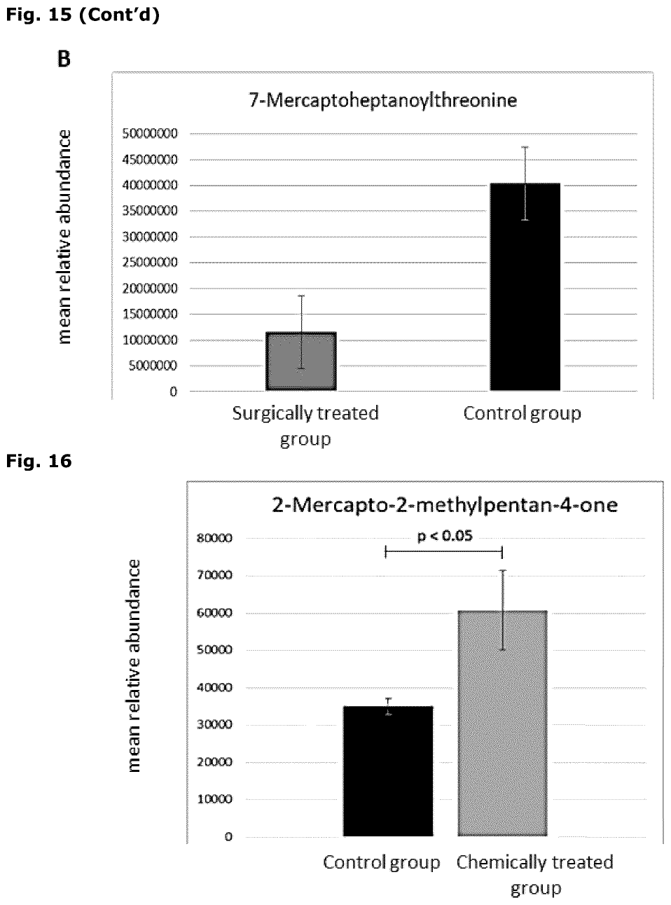

The results are reported on FIG. 15 annexed. On this figure, the mean relative abundance of 7-mercapto heptanoylthreonine present in male cat urine samples collected at D63 after administration of saline (control group) is compared with that of cats implanted with one 4.7 mg deslorelin implant (Suprelorin®) (chemically treated group) (FIG. 15A) and with that of surgically castrated male cats (surgically castrated group) (FIG. 15B).

These results show that on Day 63, 7-mercapto heptanoylthreonine was significantly decreased in the chemically treated group and in the surgically castrated group versus the control group versus the control group.

### 2-mercapto-2-methylpentan-4-one

The results are reported on FIG. 16 annexed. On this figure, the mean relative abundance of 2-mercapto-2-methylpentan-4-one present in male cat urine samples collected at D63 after administration of saline (control group) is compared with that of cats implanted with one 4.7 mg deslorelin implant (Suprelorin®) (chemically treated group).

The results demonstrate that on Day 63, 2-mercapto-2-methylpentan-4-one, known to have a rather pleasant odour of onion, was significantly increased in the chemically treated group according to the invention versus the control group.

### Crotonic Acid:

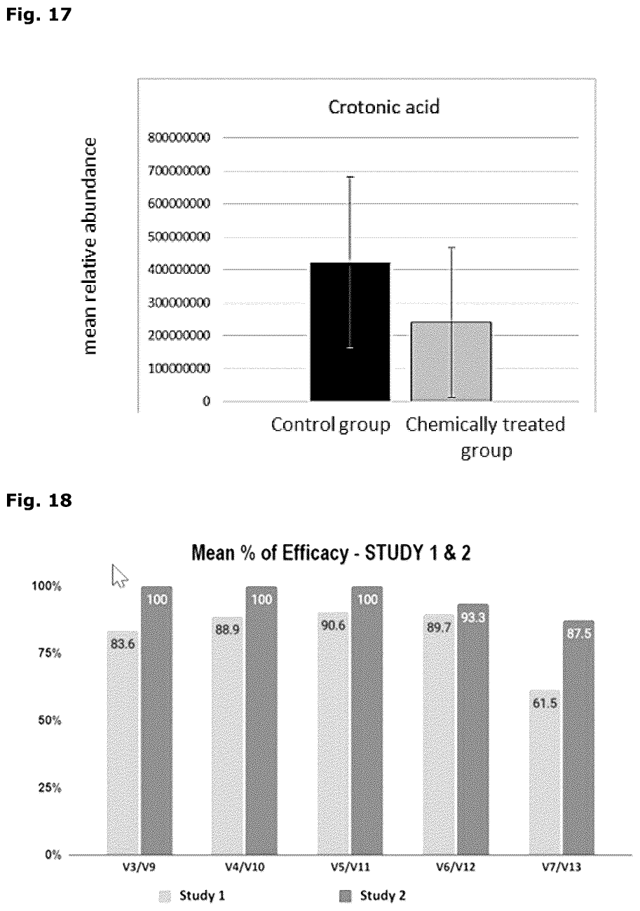

The results are reported on FIG. 17 annexed. On this figure, the mean relative abundance of crotonic acid present in male cat faeces samples collected at D63 after administration of saline (control group) is compared with that of cats implanted with one 4.7 mg deslorelin implant (Suprelorin®) (chemically treated group).

The results demonstrate that on Day 63, crotonic acid, known to have an unpleasant odour, was significantly decreased in the chemically treated group comparatively to the control group.

All results relating to the determination of mercapto derivatives in urine samples show that the concentration of mercapto derivatives in urine is influenced by physical castration and by deslorelin treatment (shown for the first time). Without willing to be bound by any theory, the Inventors of the present invention presume that these changes are not directly related to testosterone as changes differ in surgically castrated and deslorelin castrated cats (e.g. mercaptopyruvic acid). In addition, decrease in urinary odour and thus marking via the decrease in mercapto derivatives seems to be more important in deslorelin-treated cats compared to surgically castrated cats.

These results also demonstrate that other tested odorific compounds (unpleasant or pleasant odour) in urine are influenced by castration by deslorelin treatment without being influenced by surgical castration (shown for the first time). It seems that these changes are thus not directly related to the testosterone pathway as changes are different in surgically castrated and deslorelin castrated cats. In addition, decrease in urinary odour and thus marking via the decrease of other compounds than mercapto derivatives seems to be more important in deslorelin-treated cats compared to surgically castrated cats.

With these data, it was surprisingly demonstrated that deslorelin has an impact on the modulation of the levels of several metabolic molecules. In particular, deslorelin down regulate the levels of following molecules individually or together at least by two: cadaverine, indole, paracresol, isovalthine and isobuteine.

The results show that deslorelin has an impact on molecules levels linked to non-sexual hormone dependent pathway. This data show that urine and/or feces odor is also dependent of molecules that are not linked to sexual-hormone pathway.

In conclusion, it is very interesting to notice that molecules responsible for unpleasant odors were downregulated whereas molecules responsible for more pleasant odors were upregulated.

### Example 4: Cat Field Studies

### 4.1 Protocol of the Studies

The aim of these studies was to demonstrate the suppression of fertility and persistence of infertility in cat male animals.

This example provides the data collected from 2 combined studies (Double-masked Placebo-Controlled randomized) that have been conducted sequentially according to the following schedule:
Study 1: The study 1 has been conducted on a group of 205 male cats divided into two groups, one Control Group that received 1 mL of saline solution (n=51-subcutaneous administration) and one Treated Group (n=154) that received an implant of Suprelorin® 4.7 mg (subcutaneous implant-deslorelin acetate).
Study 2: At the end of a first period of 12 months, the Suprelorin® implant was removed in all the male cats belonging to the Treated Group. 12 male cats of the Treated Group issued from study 1 were then administered saline solution (1 mL subcutaneously) (Saline Group) and 22 cats of the Treated Group issued from Study 1 were re-implanted with a new Suprelorin® 4.7 mg implant for a second period of time of 12 months (Treated twice Group). The efficacy and safety of the re-implantation was followed-up for these supplementary 12 months, i.e. for a total of 24 months.

The observation schedule was as follows:
Study 1: V1: Day −14 to −7; V2: Day 0; V3: 1.5 months±3 days; V4: 3 months±5 days, V5: 6 months±5 days, V6: 9 months±5 days; V7: 12 months±5 days;
Study 2: V8: 12 months±3 days; V9: 13.5 months±3 days; V10: 15 months±5 days; V11: 18 months±5 days; V12: 21 months±5 days; V13: 24 months±5 days.

Regarding the enrolment criteria, in both studies the criteria were as the following:
Domestic Cats
Age >3 MONTHS old or >15 MONTHS in Study 2
Intact Indoor males
Client-owned (including shelter cats/rehomed by foster families)

With the additional criteria for study 2 of being a success in study 1.

In these studies the following primary endpoints were measured:
For Study 1: the suppression of fertility (Testosterone≤0.10 ng/ml*from V4 until V7*-3 to 12 months)
Study 2: the suppression of fertility (Testosterone≤0.10 ng/ml*from V8 until V13 (12 to 24 months) and the time to reversion effect.

In addition to the safety parameters (Haematology and blood biochemistry, Urinalysis, Local reactions at the implantation site, Change in Body weight, Appetite, Abnormal testicular evaluation, Adverse events), the following secondary endpoints have been reported as well:
Sexual behaviour sum score
Aggression
Vocalization
Urine marking
Urine Smell/odour
Penile Spines
Testicular volume

### 4.2 Results

### Testosterone:

The mean of percentages of efficacy results are presented in FIG. 18. Overall, the success rate of both studies was very similar for 1 or 2 implants with an Efficacy >87% at all time points in Study 2.

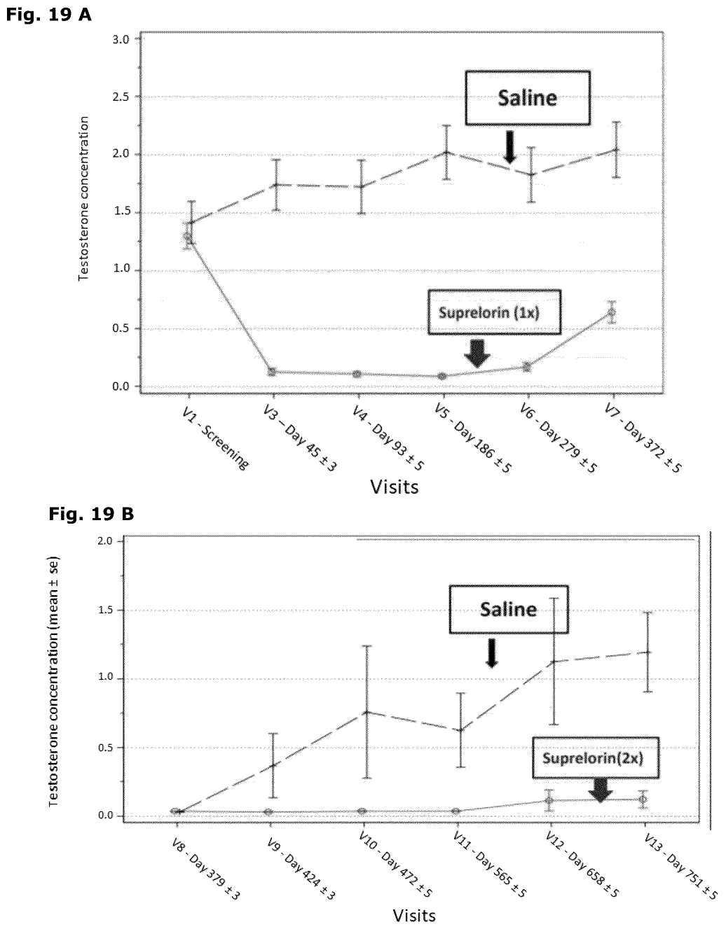

Detailed levels of Testosterone are respectively presented in FIG. 19A for study 1 and in FIG. 19B for study 2.

### The Reversibility:

The reversibility of deslorelin effect has been studied. Indeed, among the deslorelin Treated Group of study 1, n=12 subject were enrolled in the Saline group for study 2 as explained above in point 4.1.

The results showed that 100% of the cats issued from the Treated Group of Study 1 and enrolled in the Saline Group of Study 2 reversed at D279 after being enrolled in Study 2. Median time for reversibility based on serum testosterone concentration measurement was D379±178.

### The Secondary Endpoints:

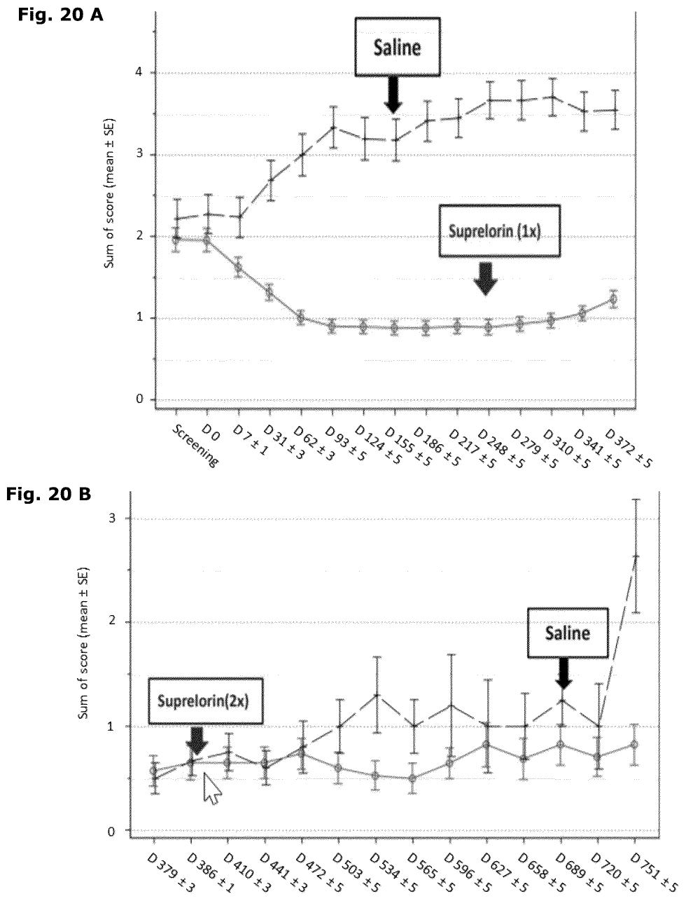

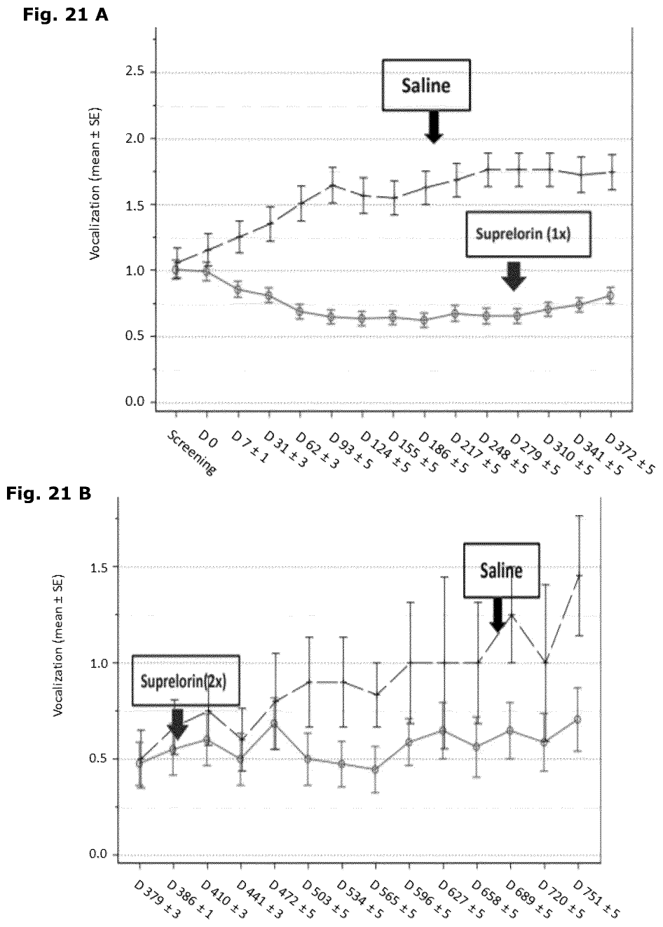

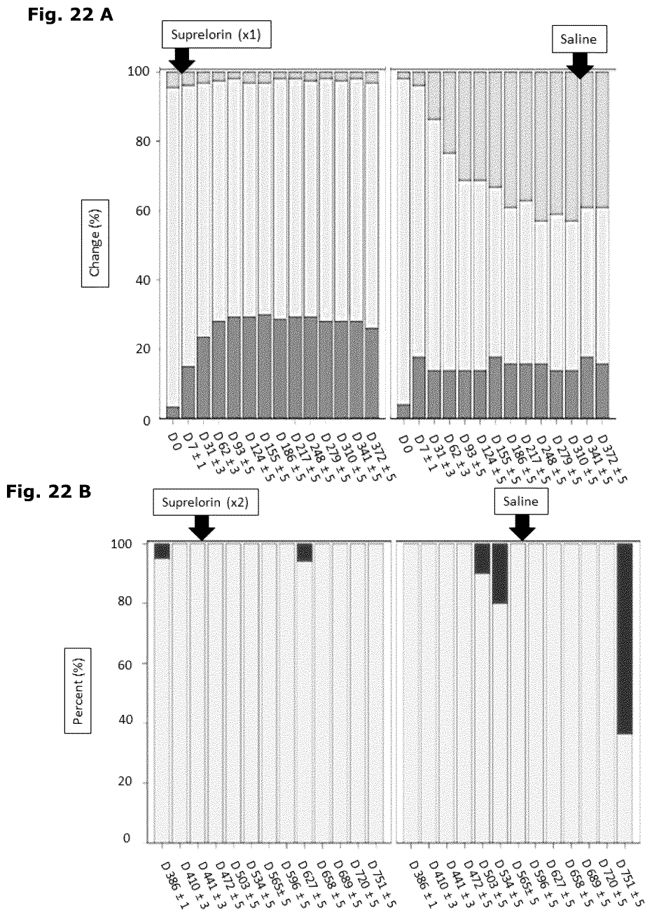

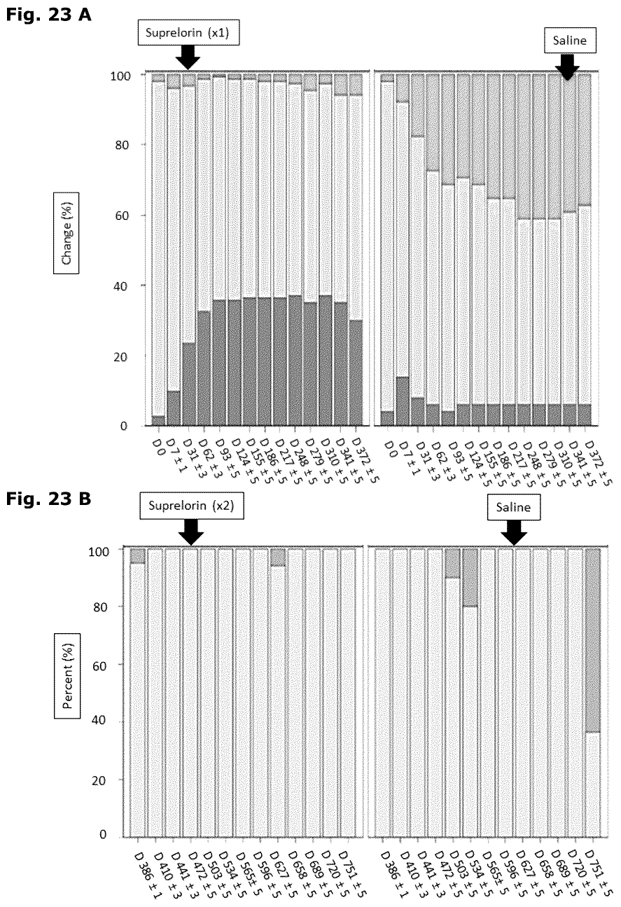

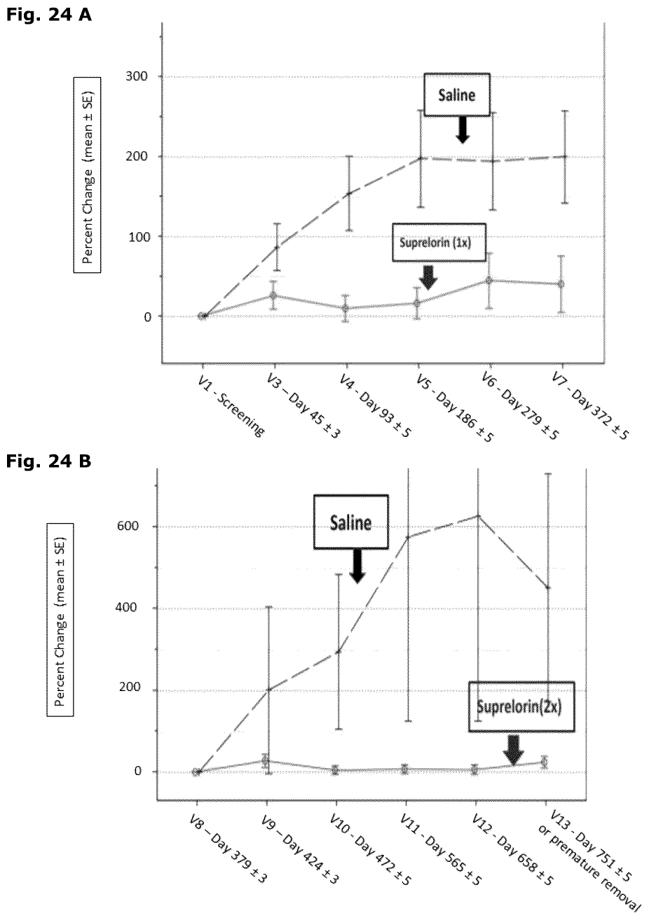

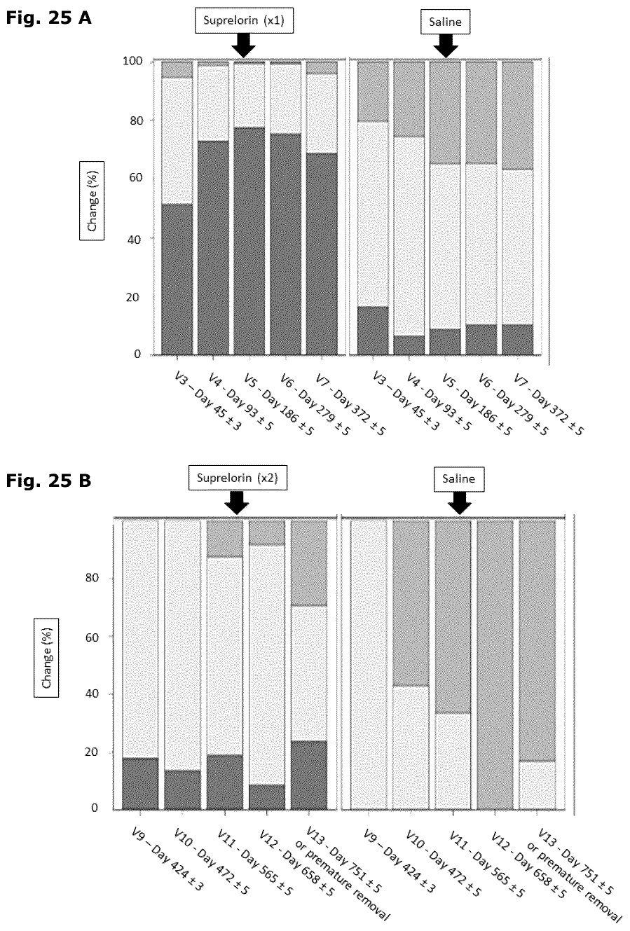

The efficacy results of the secondary endpoints have been reported with:
the mean of sum score of sexual behaviors respectively presented in FIG. 20A for study 1 and 20B for study 2,
the mean vocalization variation in FIG. 21A for study 1 and 21B for study 2,
the urine marking, the % change from baseline is respectively presented for study 1 in FIG. 22A and for study 2 in FIG. 22B,
the urine odor variation/reduction measured as the change from baseline are presented respectively for study 1 in FIG. 23A and for study 2 in FIG. 23B
the % change of testicular volume is presented respectively for study 1 in FIG. 24A and for study 2 in FIG. 24B
the % change of appearance penile spines is presented respectively for study 1 in FIG. 25A and for study 2 in FIG. 25B

During the course of the studies, there were no safety concerns and the deslorelin implants were well tolerated.

In conclusion, these combined studies have demonstrated that:

The deslorelin was effective for at least 12 months in chemically castrating the subjects. This effect has been proved to be reversible at the end of the treatment course of action. These studies have also demonstrated that sexual behaviors have been reduced with a quick onset of efficacy in behavioral reduction (approx. 7-31 days). In addition, there has been no safety concern either with the single administration or with the repeated administration.

Therefore, it can be concluded that deslorelin administered with a slow release and particularly with an implant is a good alternative to the surgical castration and is a safe non-surgical method for controlling male cat reproduction.

The method of use deslorelin as described herein is a reliable, reversible, non-surgical neutering solution to foster pet family wellbeing while keeping options open.

1-15. (canceled)

16. A method for attenuating or reducing urine and/or feces odor in a non-human animal, said process comprising administering to the non-human animal a quantity of deslorelin wherein after administration to said non-human animal, the deslorelin plasma concentration of said non-human animal is higher than 10 pg/mL.

17. The method according to claim 16, wherein after administration to said non-human animal, the deslorelin plasma concentration is between 10 pg/mL and 400 pg/ml, from 8 days after administration up to at least 6 months.

18. The method according to claim 17, wherein deslorelin is administered or released after administration continuously in said non-human animal.

19. The method according to claim 18, wherein after administration, the deslorelin is released substantially continuously from 8 days after administration to at least 6 months at a level plasma concentration below 400 pg/mL.

20. The method according to claim 18, wherein after administration, the deslorelin down regulates or up regulates the molecules responsible of urine and/or feces odor, said molecules being selected from the group consisting of felinine derivative compounds, the volatile organic compounds (VOCs), the sulfur compounds and the testosterone induced compounds.

21. The method according to claim 20, wherein after administration, the deslorelin down regulates molecules responsible of urine and/or feces odor, said molecules being selected from the group consisting of:
sulfur compounds selected from the group consisting of: 4-methyl-4-sulfanyl-pentan-2-one; 3-methyl-3-sulfanyl-butan-1-ol; and 3-methyl-3-sulfanyl-1-butyl formate;
VOCs: 2,5-dimethyl pyrazine; 1,2-dichloro-propane; 4,4-dimethyl-2-pentanone; and 3-methyl-2-buten-1-ol;
compounds selected from the group consisting of 2-(methylthio)-1-ethanol; 2-methyl-3-furanthiol; oxathiolane; 4-(methylthio)-2-butanone; 3-methylbutyl-3-sulfanyl formate; and 3-methyl-3-(methylthio)-1-butanol;
paracresol;
felinine and derivatives thereof selected from the group consisting of: 3-mercapto-3-methyl-1-butanol; 3-mercapto-3-methylbutyl formate; 3-methyl-3-methylthio-1-butanol; and 3-methyl-3-(2-methyldisulfanyl)-1-butanol;
felinine precursors selected from N-acetylfelinine, γ-glutamylfelinylglycine and 3-methylbutanolcysteinylglycine;
cauxine;
2-phenylethylamine, and S-(1-hydroxy-3,7-dimethyl-6-octen-3-yl)cysteine;
isovalthine;
isobuteine;
trimethylamine;
indole;
cadaverine and derivatives selected from the group consisting of N-acetylcadaverine, and glutathionylaminopropylcadaverine;
short-chain free fatty acids selected from the group consisting of: acetic acid, propanoic acid, 2-methylpropanoic acid, butanoic acid, 3-methylbutanoic acid, and pentanoic acid;
crotonic acid;
putrescine and derivatives thereof selected from the group consisting of N-acetylputrescine, and p-coumaroylputrescine;
mercapto derivatives selected from the group consisting of 2-mercapto-3-butanone, 2-mercaptoethanol, 3-mercaptohexyl butyrate, mercaptopyruvic acid, 3-mercaptopropionic acid, 3-mercapto-3-methylbutan-1-ol, 7-mercaptoheptanoylthreonine, 1alpha,5alpha-Dimercaptoandrostane-3alpha,17beta-diol, mercaptopyruvic acid, and 2-mercapto-2-methylpentan-4-one; and
taurine.

22. The method according to claim 21, wherein said molecules are selected from the group consisting of indole, N-acetylcadaverine, putrescine and derivates thereof selected from the group consisting of N-acetylputrescine and p-coumaroylputrescine, and mercaptopyruvic acid.

23. The method according to claim 20, wherein after administration, the deslorelin up regulates molecules responsible of urine and/or feces odor, said molecules being selected from the group consisting of 2-mercapto 2-methylpentan-4-one and valine.

24. A method to down regulate the levels of molecules in urine and or feces of a non-human animal, wherein said process comprises a step of administering to the non-human animal a quantity of deslorelin and wherein said molecules are selected from the group consisting of:
sulfur compounds selected from the group consisting of: 4-methyl-4-sulfanyl-pentan-2-one; 3-methyl-3-sulfanyl-butan-1-ol; and 3-methyl-3-sulfanyl-1-butyl formate;
VOCs selected from the group consisting of: 2,5-dimethyl pyrazine; 1,2-dichloro-propane; 4,4-dimethyl-2-pentanone; and 3-methyl-2-buten-1-ol;
compounds selected from the group consisting of 2-(methylthio)-1-ethanol; 2-methyl-3-furanthiol; oxathiolane; 4-(methylthio)-2-butanone; 3-methylbutyl-3-sulfanyl formate; and 3-methyl-3-(methylthio)-1-butanol;
paracresol;
felinine and derivatives thereof selected from the group consisting of: 3-mercapto-3-methyl-1-butanol; 3-mercapto-3-methylbutyl formate; 3-methyl-3-methylthio-1-butanol; and
3-methyl-3-(2-methyldisulfanyl)-1-butanol;
felinine precursors selected from the group consisting of: N-acetylfelinine, γ-glutamylfelinylglycine and 3-methylbutanolcysteinylglycine;
cauxine;
2-phenylethylamine, and S-(1-hydroxy-3,7-dimethyl-6-octen-3-yl)cysteine;
isovalthine;
isobuteine;
trimethylamine;
indole;
cadaverine and cadaverine derivatives selected from the group consisting of N-acetylcadaverine, and glutathionylaminopropylcadaverine;
short-chain free fatty acids selected from the group consisting of: acetic acid, propanoic acid, 2-methylpropanoic acid, butanoic acid, 3-methylbutanoic acid, and pentanoic acid;
crotonic acid;
putrescine and derivatives thereof selected from the group consisting of N-acetylputrescine, and p-coumaroylputrescine;
mercapto derivatives selected from the group consisting of 2-mercapto-3-butanone, 2-mercaptoethanol, 3-mercaptohexyl butyrate, mercaptopyruvic acid, 3-mercaptopropionic acid, 3-mercapto-3-methylbutan-1-ol, 7-mercaptoheptanoylthreonine, 1 alpha,5alpha-dimercaptoandrostane-3alpha,17beta-diol, mercaptopyruvic acid, and 2-mercapto-2-methylpentan-4-one; and
taurine.

25. A method to up regulate the levels of molecules in urine and/or feces of a non-human animal, wherein said method comprises a step of administering to the non-human animal a quantity of deslorelin and wherein said molecules being selected from the group consisting of 2-mercapto 2-methylpentan-4-one and valine.

26. A method for attenuating or reducing urine and/or feces odor in a non-human animal, the process comprising administering to the non-human animal a quantity of deslorelin that will induce down regulations or up regulations of the levels of odorifying molecules in urine and/or feces.

27. The method according to claim 26, wherein the non-human animal is an intact male cat.

28. The method according to claim 27, wherein deslorelin induces down regulations or up regulations of the levels of odorifying molecules in urine and/or feces via sex hormone synthesis pathway.

29. The method according to claim 26, wherein deslorelin induces down regulations or up regulations of the levels of odorifying molecules in urine and/or feces via sex hormone synthesis pathway.

30. A method for improving the attenuation or reduction of the urine and/or feces odor in a non-human intact male animal compared to a neutered male, the process comprising administering to the non-human intact male animal a quantity of deslorelin that induces down regulations or up regulations of the levels of odorifying molecules in urine and/or feces.
